SEQUENCE LISTING (30 sequences)

SEQ ID NO: 1 - RNA, 21 nt, Homo sapiens
caacaccagucgaugggcugu

SEQ ID NO: 2 - RNA, 22 nt, Mus musculus
caacagcagucgaugggcuguc

SEQ ID NO: 3 - DNA, 21 bp, Artificial Sequence
  complementary sequence of human miR-21-3p
acagcccatcgactggtgttg

SEQ ID NO: 4 - DNA, 15 bp, Artificial Sequence
  Human miR-21-3p inhibitor 1
ccatcgactggtgtt

SEQ ID NO: 5 - DNA, 18 bp, Artificial Sequence
  Human miR-21-3p inhibitor 2
agcccatcgactggtgtt

SEQ ID NO: 6 - DNA, 21 bp, Artificial Sequence
  complementary sequence of murine miR-21-3p
acagcccatcgactgctgttg

SEQ ID NO: 7 - DNA, 15 bp, Artificial Sequence
  Murine miR-21-3p inhibitor 1
ccatcgactgctgtt

SEQ ID NO: 8 - DNA, 18 bp, Artificial Sequence
  Murine miR-21-3p inhibitor 2
agcccatcgactgctgtt

SEQ ID NO: 9 - DNA, 15 bp, Artificial Sequence
  basic sequence of mmu-miR-21a-3p_3MM without FAM labelling
ccctagactgctctt

SEQ ID NO: 10 - DNA, 21 bp, Artificial Sequence
  mmu-Gapdh mRNA forward primer
gtatgactccactacggcaaa

SEQ ID NO: 11 - DNA, 17 bp, Artificial Sequence
  mmu-Gapdh mRNA reverse primer
ttcccattctcggcttg

SEQ ID NO: 12 - DNA, 18 bp, Artificial Sequence
  mmu-Ef1a1 mRNA forward primer
cctggcaagcccatgtgt

SEQ ID NO: 13 - DNA, 21 bp, Artificial Sequence
  mmu-Ef1a1 mRNA reverse primer
tcatgtcacgaacagcaaagc

SEQ ID NO: 14 - DNA, 17 bp, Artificial Sequence
  mmu-Ppar beta/delta mRNA forward primer
cggcagcctcaacatgg

SEQ ID NO: 15 - DNA, 23 bp, Artificial Sequence
  mmu-Ppar beta/delta mRNA reverse primer
agatccgatcgcacttctcatac

SEQ ID NO: 16 - DNA, 23 bp, Artificial Sequence
  hsa-Ppar beta/delta mRNA forward primer
gcatgaagctggagtacgagaag

SEQ ID NO: 17 - DNA, 20 bp, Artificial Sequence
  hsa-Ppar beta/delta mRNA reverse primer
gcatccgaccaaaacggata

SEQ ID NO: 18 - DNA, 17 bp, Artificial Sequence
  mmu-Ppar beta/delta mRNA forward primer
cggcagcctcaacatgg

SEQ ID NO: 19 - DNA, 23 bp, Artificial Sequence
  mmu-Ppar beta/delta mRNA reverse primer
agatccgatcgcacttctcatac

SEQ ID NO: 20 - DNA, 20 bp, Artificial Sequence
  hsa-P21 mRNA forward primer
ctgtcactgtcttgtaccct

SEQ ID NO: 21 - DNA, 21 bp, Artificial Sequence
  hsa-P21 mRNA reverse primer
ggtagaaatctgtcatgctgg

SEQ ID NO: 22 - DNA, 21 bp, Artificial Sequence
  hsa-P15 mRNA forward primer
cgtgggaaagaagggaagagt

SEQ ID NO: 23 - DNA, 15 bp, Artificial Sequence
  hsa-P15 mRNA reverse primer
ccccagacgcgcagc

SEQ ID NO: 24 - DNA, 20 bp, Artificial Sequence
  hsa-pri-miR-21 forward primer
ttttgttttgcttgggagga

SEQ ID NO: 25 - DNA, 20 bp, Artificial Sequence
  hsa-pri-miR-21 reverse primer
agcagacagtcaggcaggat

SEQ ID NO: 26 - DNA, 27 bp, Artificial Sequence
  basic sequence of miR-21-3p LNA probe without Dig labelling
gacagcccatcgactgcactgctgttg

SEQ ID NO: 27 - DNA, 21 bp, Artificial Sequence
  basic sequence of Scramble-miR LNA probe without Dig labelling
gtgtaacacgtctatacgcca

SEQ ID NO: 28 - RNA, 24 nt, Homo sapiens
ucacaaccuccuagaaagaguaga

SEQ ID NO: 29 - DNA, 16 bp, Artificial Sequence
  Human miR-21-3p inhibitor “I-has-miR-21-3p
cccatcgactggtgtt

SEQ ID NO: 30 - DNA, 16 bp, Artificial Sequence
  Human miR-21-3p mismatch control ”MM-has-miR-21-3p“
cccttcgtcaggtgta

(19) United States
(12) Patent Application Publication
(10) Pub. No.: US 2017/0175112 A1
(43) Pub. Date: Jun. 22, 2017

(54) MIR-21-3P INHIBITORS IN SKIN DISORDERS

(71) Applicant: UNIVERSITE DE LAUSANNE, Lausanne (CH)

(72) Inventors: Gwendoline DEGUEURCE, Lausanne (CH); Ilenia D'ERRICO, Lausanne (CH); Liliane MICHALIK, Bussigny (CH); Walter WAHLI, Echichens (CH); Alexandra MONTAGNER, Toulouse (FR)

(73) Assignee: UNIVERSITE DE LAUSANNE, Lausanne (CH)

(21) Appl. No.: 15/118,374

(22) PCT Filed: Feb. 13, 2015
(86) PCT No.: PCT/EP2015/053043

§ 371 Date: Aug. 11, 2016

(30) Foreign Application Priority Data
Feb. 14, 2014 (EP) 14155184.6

(51) Int. Cl. C12N 15/113; C12Q 1/68

(52) U.S. Cl. CPC C12N 15/113; C12Q 1/6883; C12N 2310/113; C12N 2310/3231; C12N 2310/3515; C12N 2310/321; C12N 2310/315; C12Q 2600/118; C12Q 2600/158

(57) ABSTRACT

The present invention is related to miR-21-3p inhibitors, which are particularly useful in the prevention and/or treatment of skin disorders.

### FIELD OF THE INVENTION

The present invention relates to miR-21-3p inhibitors useful for treatment of skin disorders.

### BACKGROUND OF THE INVENTION

Psoriasis is a chronic inflammatory skin disease affecting 1 to 3% of the Caucasian population with a considerable negative impact on the patient's quality of life. This disease causes red scaly patches on the skin that are areas of inflammation. Over time, affected skin can become resistant to treatment, especially when topical corticosteroids are used.

MicroRNAs (miRNAs) are endogenous small RNAs which can target messenger RNAs (mRNAs) and generate their degradation or prevent their translation. Before complete maturation of miRNA, the endoribonuclease DICER1 generates a double-stranded RNA fragment constituted of miRNA-5p and miRNA-3p strands (the guide strand and the passenger strand). The guide strand is selected by an RNase Argonaute on the basis of the thermodynamic stability of the 5′-end. While the regulatory functions of the guide strands loaded into the RISC complex have been largely described, passenger strands were thought to be degraded and their functions have been largely discounted.

MiRNAs are involved in several human pathologies (Bartel, 2004, Cell, 116(2):281-97; Alvarez-Garcia et al., 2005, Development, 132(21):4653-62) as well as in skin development, homeostasis and disease (Schneider, 2011, Br. J. Dermatol., 166(1):22-8).

Up till now few studies have analyzed the expression of microRNAs in the skin. One study showed that the microRNA “miR-203” is highly expressed in the skin and overexpressed in psoriatic skin, in addition expression of another microRNA “mature miR-21” is increased in both psoriasis and atopic eczema (Sonkoly et al., 2007, PLoS One, 2(7): e610). Meisgen et al. (Exp Dermatol, 2012, 21(4):312-4) showed that miR-21-5p (commonly named miR-21) is upregulated in psoriasis and different T cell types (that leads to T cell apoptosis suppression). Efficient inhibition of miR-21 performed only in primary human T helper cells was achieved by using seed-targeting 8-mer locked nucleic acid (LNA) oligonucleotides, termed tiny LNAs, and resulted in decrease of T cell apoptosis rate (Meisgen et al., 2012, supra). Syed et al., 2013, Curr Drug Targets, 14(10): 1128-34) demonstrated involvement of a few microRNAs, including miR-21-5p in skin response to UV radiation.

Recently, expression of miR-21-3p was demonstrated to be higher in tissues and serum among non-small cell lung carcinoma patients compared to healthy volunteers (Jiang et al., 2013, Mol Cell Biochem, 383:67-75). Aure et al., 2013, Genome Biology, 14:R126 studied individual and combined effects of DNA methylation and copy number alterations on miRNA expression in breast tumors. Their results suggest that miR-21-3p may play an oncogenic role in breast cancer. Lo et al., 2013, PLoS One, 8(9):e75628 showed that miR-21-3p inhibits hepatoma cell growth and functions as a tumor suppressor in hepatocellular carcinoma.

Despite growing interest and efforts in developing therapeutics from miRNAs, there remains a need for new therapeutic treatments for skin disorders including, in particular, inflammatory skin disorders, UV damages, skin cancers.

### SUMMARY OF THE INVENTION

The present invention is mainly directed towards miR-21-3p inhibitors and the use thereof, in particular in the prevention and/or treatment of skin disorders.

A first aspect of the invention provides miR-21-3p inhibitors.

A second aspect of the invention relates to compositions comprising miR-21-3p inhibitors.

A third aspect of the invention relates to miR-21-3p inhibitors for use as a medicament.

A fourth aspect concerns the use of inhibitors of miR-21-3p for the preparation of a medicament.

A fifth aspect concerns a pharmaceutical preparation comprising miR-21-3p inhibitors or a vector comprising a nucleic acid encoding said miR-21-3p inhibitors, and a pharmaceutically acceptable carrier.

A sixth aspect relates to miR-21-3p inhibitors for use in the prevention and/or treatment of skin disorders.

A seventh aspect provides the use of said miR-21-3p inhibitors for the manufacture of a medicament for the prevention and/or treatment of skin disorders.

An eighth aspect of the invention relates to a method of preventing and/or treating skin disorders, said method comprising administering in a subject in need thereof a miR-21-3p inhibitor, or a pharmaceutical formulation thereof.

A ninth aspect of the invention relates to an ex vivo method of prognosis and/or diagnosis of a skin disorder in a subject comprising determining, in a biological sample of said subject, the level of miR-21-3p.

Other features and advantages of the invention will be apparent from the following detailed description.

### DESCRIPTION OF THE FIGURES

FIG. 1 shows that PPARβ/δ regulates the expression of UV-induced epidermal miR-21-3p, a miRNA also upregulated in human skin carcinomas. A: miR-21-3p/miR-21-5p ratio: number of miR-21-3p reads on the number of miR-21-5p reads obtained from RNA sequencing in mouse total skin, kidney, testis, brain and heart. B: RT-qPCR quantification of relative miR-21-3p levels in total skin of chronically irradiated (Chr-UV) and non-irradiated (no UV) PPARβ/δ+/+ (black) and PPARβ/δ−/− (grey) mice. (*) p value<0.01, (ns) for non-significant, N=4. C: RT-qPCR quantification of relative miR-21-3p levels in human normal skin (black) and squamous cell carcinomas (SCC, grey), (*) p value<0.01, N=3. D: miR-21-3p in situ hybridization (light signal, also indicated by the arrows) in the skin of acutely irradiated (Ac-UV) (bottom line) and no UV (upper line) PPARβ/δ+/+ (left hand column) and PPARβ/δ−/− (right hand column) mice. E: RT-qPCR quantification of relative miR-21-3p level in the epidermis of Ac-UV and no UV PPARβ/δ+/+(black) and PPARβ/δ−/− (grey) mice, (*) p value<0.01, N=4. F: RT-qPCR quantification of relative miR-21-3p levels in HaCat cells treated with the PPARβ/δ agonists GW501516 or GW0742, (*) p value<0.01, N=3.

FIG. 2 shows that inflammatory markers are up-regulated in miR-21-3p mimic treated HaCat keratinocytes. Relative mRNA level of Cox2 (A) and IL6 (B) determined by real-time qPCR quantification (miR-21-3p mimic—white bar, control—black bar), (*) p value<0.05, N=3.

FIG. 3 shows that phosphatidylcholine content is up-regulated in miR-21-3p mimic treated HaCat keratinocytes. Bars indicate the fold changes in mimic treated HaCat keratinocytes compared to control treated HaCat keratinocytes.

FIG. 4 shows that the cell cycle inhibitors p15 (B) and p21 (A) are down regulated in miR-21-3p mimic transfected HaCat keratinocytes (white) compared to control (black). (*) p value<0.05, N=3.

FIG. 5 shows that MMP1 is down-regulated in miR-21-3p mimic treated HaCat keratinocytes (white) compared to control (black). (*) p value<0.05, N=3.

FIG. 6 shows that miR-21-3p inhibitor fluorescence increases in the skin upon UV-induced inflammatory condition. Ratio of miR-21-3p fluorescence (FAM) on control fluorescence value quantified in skin, liver and spleen of acutely irradiated (Ac-UV, white) or non-irradiated (no UV, black) mice, N=4.

FIG. 7 shows a gain of miR-21-3p function provokes changes in inflammation pathways in HaCaT human keratinocytes. A: Go term enrichment analysis based on the significantly up and down regulated mRNA in miR-21-3p mimic treated HaCaT cells compared to control treated cells. RT-qPCR of I16 (B), I11α (C), I11RAP (D), Cox2 (E), Ccl5 (F), Cxc10 (G) and Casp14 (H) levels in HaCaT cells transfected with a miR-21-3p mimic (black) or a scramble sequence (grey); (*) p value<0.05, N=3.

FIG. 8 shows that miR-21-3p is not up-regulated in non-inflamatory situation. A: RT-qPCR of miR-21-3p level in murine skin after tape stripping (Tape stripping skin, TS) or without any stress (Normal skin, NS), N=4. RT-qPCR of I11β (B) and Tnfα (C) levels in murine skin after tape stripping (Tape stripping skin, TS) or without any stress (Normal skin, NS), N=4.

FIG. 9 demonstrates preventive anti-inflammatory effect of human miR-21-3p inhibitor after UV exposure of er vive cultured human abdominal skin. RT-qPCR of Cox2 (A) and I16 (B) mRNAs on cultured ex vivo normal abdominal skin topically treated with the miR-21-3p-based formulation (miR-21-3p inhibitor) or with a mismatch control based formulation (CTR) prior and immediately after UV irradiation (grey) or not UV irradiated (black). (*) p value<0.05, N technique=4, N. patient=1.

FIG. 10 shows that MIR-21-3p is up-regulated under skin inflammatory conditions: human psoriasis biopsies. RT-qPCR quantification of relative pri-miR-21 (A), miR-21-5p (B) and miR-21-3p (C) levels in normal human skin (black) and human psoriatic lesional skin (grey), (*) p value<0.01, N≧4

FIG. 11 shows MIR-21-3p involvement in human cell melanoma and in keratinocytes migration. A: RT-qPCR quantification of relative human miR-21-3p expression level in benign melanocytic naevus (BMN), in primary melanoma (PM) and in malignant melanoma (MM) (*) p value<0.05, (****) p value<0.001. B. RT-qPCR quantification of relative human miR-21-3p metastatic melanoma cell lines (A375, SKMe128 and WM983B) compared with normal melanocytes (NHM) and with non-metastatic melanoma cell lines (WM35, WM115 and A375), (*) p value<0.05.

FIG. 12 shows that MMP1 is a direct target of miR-21-3p and that miR-21-3p levels are correlated with wound healing process. A: In silico predicted miR-21-3p seed sequences on Human MMP1 3′UTR, genomic position, seed type (“X:Y:Z” notation is for the size of the seed (X), the number of mismatches (Y) and the number of G:U wobble pairs (Z) and ΔΔG (ΔΔG is an energetic score, the lower (more negative) its value, the stronger the binding of the microRNA to the given site is expected to be. The ΔΔG is an energetic score is based on the PITA algorithm, which considers the role of target-site accessibility. Reference for the Pita algorithm: Kertesz et al., 2007, Nature Genetics, 39(10): 1278-1284. B: Luciferase signal related to MMP1 3′UTR (3′UTR MMP1-Luc) and Luciferase signal related to mutated MMP1 3′UTR (Mut-MMP1-Luc, point mutations are in boxes) related to transcriptional activity in miR-21-3p mimic transfected NHEK cells or in scramble treated cells (CTR), (**) p value<0.01. C: RT-qPCR of miR-21-3p expression level before (day 0), during (day 1 to day 7), and after (day 10), the skin wound healing process, (*) p value<0.05, (**) p value<0.01.

### DETAILED DESCRIPTION OF THE INVENTION

The terms “Micro-RNA”, “miRNA”, and “miR” designate herewith a class of 17-25 nucleotides non-coding single stranded RNA molecules that regulate the expression of target RNA by either translational inhibition or mRNAs degradation. Animal miRNAs typically exhibit only partial complementarity to their mRNA targets. A ‘seed region’ of about 6-8 nucleotides in length at the 5′ end of an animal miRNA is thought to be an important determinant of target specificity. In animals, miRNAs are generated from primary transcripts (pri-miRNAs) generally transcribed from polymerase-II (Pol-II) promoters and processed in the nucleus by Drosha enzyme to about 60-70 nucleotides stem-loop (s1) molecules with two-nucleotide 3′ overhangs (pre-miRNA). Pre-miRNAs are shuttled by exportin 5 to the cytosol where Dicer enzyme releases an about 21-24 double-stranded RNA from the stem. This is loaded into the RNA-induced silencing complex (RISC), which generally selects one of the two strands as the guide strand (mature miRNA), according to thermodynamic properties. The other strand, called the passenger strand, was generally thought, up till recently, to be degraded and its function has been largely discounted. RISC targets mRNA with complementary sequence to the miRNA and downregulates their expression decreasing transcript translation and stability by a variety of molecular mechanisms.

“MicroRNA-21-3p”, “miRNA-21-3p”, “miR-21-3p”, “microRNA-21*”, “miR-21*” or “miR21*”, designate the passenger strand of the pre-miRNA-21, the guide strand of said pre-miRNA being the miR-21-5p. The nucleic acid sequence of human miR-21-3p is represented under SEQ ID NO: 1 (miRBase Accession number MIMAT0004494). The nucleic acid sequence of murine miR-21-3p is represented under SEQ ID NO: 2 (miRBase Accession number MIMAT0004628). Human and murine miR-21-3p sequences differ only by two nucleotides. Murine miR-21-3p sequence is longer by 1 base at the extremity 5′ as compared to its human homologue. The term “miR-21-3p” covers the different variants of miR-21-3p of different mammalian species. As used herewith the term “miR-21-3p” covers the mature miR-21-3p, as well as the region, within the pri-miRNA-21 and pre-miRNA-21, having a nucleotide sequence identical to that of miR-21-3p and in particular, having nucleotide sequence comprising the minimal functional sequence (seed region) from nucleotide of position 2 to 8 of SEQ ID NO: 1, and more particular a functional sequence containing nucleotide from position 2 to 16 of SEQ ID NO: 1 (extended seed region).

The term “variant of a nucleic acid” as referred to herein, includes a nucleic acid (e.g. RNA or DNA) substantially homologous to the original nucleic acid sequence, but which has at least one nucleotide different from that of the original sequence because of one or more deletions, insertions or substitutions. Substantially homologous means a variant nucleic acid sequence that is at least 80%, at least 85%, at least 90%, at least 95%, at least 96%, at least 97%, at least 98% or at least 99% identical to the original nucleic acid sequence. The variant of a nucleic acid that is an inhibitor of a miR-21-3p as defined herewith is capable of inhibiting the function of said miR-21-3p. According to one aspect, the variant of a nucleic acid that is an inhibitor of a miR-21-3p typically differs from the specified nucleic acid sequence by 2 nucleotides, in particular by 1 nucleotide.

The terms “Percentage of identity”, “% identity”, or the like, refer to the level of identity between two nucleic acid sequences. The percentage of identity of two sequences can be determined by visual inspection and/or mathematical calculation, or more easily by comparing sequence information using known computer program used for sequence comparison such as Clustal package version 1.83.

The term “to hybridize” or “hybridizing” means annealing nucleic acid strands from different sources, i.e. forming base pairs between complementary regions of two strands of nucleic acids that were not originally paired, for instance under stringent conditions. Typically, “hybridization under stringent conditions” as defined according to Sambrook et al. Molecular Cloning, A Laboratory Manual, Cold Spring Harbor, Laboratory Press (1989), 1.101-1.104) is best obtained with fully complementary regions of two strands of nucleic acids. However, depending on various parameters such as the length of the regions of the nucleic acids to be hybridized and their specific nucleotide sequences, a few mismatches may be tolerated without substantially impairing hybridization. In particular embodiments, two nucleic acids are considered to hybridize to each other when the percentage of identity between one of the nucleic acid sequence and the complementary sequence of the other nucleic acid sequence is at least 80%, at least 85%, at least 90%, at least 95%, at least 96%, at least 97%, at least 98% or at least 99%. The expression “inflammatory skin disorders” as defined herewith designates disorders affecting the skin. This includes skin infections and skin neoplasms, including skin cancers.

The expression “Inflammatory skin disorders” as defined herewith designates disorders affecting the skin accompanied by an inflammatory process. This includes psoriasis, dermatitis, acne, rosacea, Stevens-Johnson syndrome, toxic epidermal necrolysis, systemic lupus erythematosus, skin allergies, atopic eczema, parakeratosis. Further, it also includes keratosis, vitiliago, leprosy, epidermolytic ichthyosis, dermatitis atopic eczema, skin cancers, non-pre-malignant or pre-malignant structure affecting the skin, UV skin photodamages, UV erythema, skin ageing, skin ageing following UV exposure, and wounds.

“Psoriasis” is a common, chronic immune-mediated skin disease which may also affect the joints. Psoriatic skin shows symptoms of inflammation and raised and scaly lesions. There are five main types of psoriasis: plaque, guttate, inverse, pustular, and erythrodermic. The most common form, plaque psoriasis, is commonly seen as red and white hues of scaly patches appearing on the top layer of the skin. Plaques frequently occur on the skin of the elbows and knees, but can affect any area, including the scalp, palms of hands and soles of feet, and genitals. In contrast to eczema, psoriasis is more likely to be found on the outer side of the joint. Fingernails and toenails are frequently affected (psoriatic nail dystrophy) and can be seen as an isolated sign. When psoriasis is accompanied with an inflammation of the joints, it is called “psoriatic arthritis”, which is also covered by the term “psoriasis” as defined herewith. Up to 30% of individuals with psoriasis also develop psoriatic arthritis.

The terms “dermatitis” or “eczema” designate herewith an inflammation of the skin. These terms broadly apply to a range of persistent skin conditions including dryness and recurring skin rashes that are characterized by one or more of the following symptoms: redness, skin swelling, itching and dryness, crusting, flaking, blistering, cracking, oozing, or bleeding. Main types of dermatitis include atopic dermatitis, contact dermatitis, xerotic eczema, and seborrheic dermatitis.

The expression “skin cancers” designates cancers affecting the skin, including squamous cell carcinomas, basal cell carcinomas, melanomas and metastatic melanoma.

The expressions “non-pre-malignant or pre-malignant” designate structure affecting the skin such as actinic keratosis and seborrheic keratosis.

An “inhibitor of a micro-RNA” as defined herewith designates any molecule, biologically active compound, or complex of molecules, e.g. a polypeptide, a nucleic acid, peptide nucleic acid (PNA), or a small chemical molecule, that is able to repress said micro-RNA expression and/or inhibit said micro-RNA function/activity. For instance, the function of a micro-RNA can be inhibited by a molecule such as a nucleic acid sequence complementary to the micro-RNA's seed region, by a molecule such as a nucleic acid sequence complementary to the extended micro-RNA's seed region, by a protein that can modify the miR-21-3p regulatory function such as RNA-binding proteins as described in van Kouwenhove et al., 2011, Nat. Rev. Cancer, 11(9):644-656, by target protector (TP) oligonucleotides that interfere with a single miRNA-mRNApair by binding specifically morpholinos which are antisense to the miRNAtarget sequence in the 3′ UTR (Staton et al., 2011, Nature Protocols, 6(12): 2035-2049).

The terms “short hairpin RNA”, “small hairpin RNA” and “shRNA” designate herewith a sequence of RNA that makes a tight hairpin turn that can be used to silence target gene expression via RNA interference (RNAi). Expression of shRNA in cells is typically accomplished by delivery of plasmids or through viral or bacterial vectors.

The term “small inhibitory nucleic acids” (siNAs) refers to short nucleic acids used in strategies targeting mRNA recognition and its downregulation based on their antisense action. This term covers antisense oligonucleotides, catalytic nucleic acids such as ribozymes and deoxyribozymes, as well as small interfering RNAs (siRNAs).

The term “siRNA” refers to small interfering RNA which are single or double stranded RNA (about 19-23 nucleotides) able to knock down or silence a targeted mRNA from a target gene. Artificial siRNAs can be either chemically synthesized as oligonucleotides or cloned into a plasmid or a virus vector (adenovirus, retrovirus or lentivirus) as short hairpin RNAs (shRNAs) to generate a transient or stable transfection in any type of cells (Martin et al., 2007, Ann. Rev. Genomics Hum. Genet., 8:81-108; Kolfschoten et al., 2007, Nat. Clin. Pract. Endocrinol. Metab., 3(12):827-34; Huang et al., 2008, Expert. Opin. Ther. Targets, 12(5), 637-645).

As used herewith an “antagomir” or “anti-miR” designates an antisense oligonucleotide (e.g. DNA, RNA, chimeric DNA/RNA, LNA, chimeric LNA/DNA) harboring the full or partial complementary sequence of a mature miRNA that can reduce the endogenous levels of a miRNA. This term also covers antagomirs which have been chemically modified such as for instance as described in van Rooij, 2011, Circ Res., 108:219-234). Examples of chemical modifications include 2′-O-methyl-group (OMe)-modified oligonucleotides and locked nucleic acid (LNA)-modified oligonucleotides, in which the 2′-O-oxygen is bridged to the 4′-position through a methylene linker to form a rigid bicycle, locked into a C3′-endo (RNA) sugar conformation. Another chemical modification applied to enhance oligonucleotide stability is the balance between phosphodiester and phosphorothioate linkages between the nucleotides, with phosphorothioate providing more stability to the oligonucleotide and making it more resistant to nucleases.

A “sponge vector” typically designates a vector, in particular a lentiviral vector, for overexpressing miRNA target sequences complementary to a miRNA seed region. The nucleic acid sequence comprised in the sponge vector does not need to be perfectly complementary to the miRNA seed region for inhibiting said micro-RNA function.

The term “subject” as used herein refers to mammals. For example, mammals contemplated by the present invention include human, primates, domesticated animals such as cattle, sheep, pigs, horses, laboratory rodents and the like.

The term “LNA-modified oligonucleotide” includes any oligonucleotide either fully or partially modified with LNA monomers. Thus, an LNA-modified oligonucleotide may be composed entirely by LNA monomers, or a LNA-modified oligonucleotide may comprise one LNA monomer. As used herein, the term “LNA monomer” typically refers to a nucleoside having a 2′-4′ cyclic linkage, as described in the International Patent Application WO 99/14226.

The expression “biological sample” refers to a clinical sample for testing which is taken from a mammal such as biopsy material, in particular skin samples.

The expression “control sample” refers to a negative control sample. A negative control sample includes a biological sample taken from a subject that is of the same or homologous species as the subject to be assayed for miR-21-3p and is known to have a normal biological state, e.g. without detectable symptoms of an inflammatory skin disease. A negative control sample includes a sample taken from a control subject.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it for example based on familial history; (b) inhibiting the disease, i.e., arresting its development; or relieving the disease, i.e., causing regression of the disease and/or its symptoms or conditions such as improvement or remediation of damage. Treatment of a skin disorder can be accompanied by a reduction in redness, itching, dryness, and/or swelling, for instance. In particular, treatment of psoriasis can be accompanied by a reduction of patches, papules or plaques on the skin. In particular, treatment of psoriasis may include prevention or delay of its relapses events or rebound. Treatment of a skin cancer can be accompanied by a decrease or eradication of the cancer stem cell populations which are at the origin of the tumor, tumor growth, recurrence and metastasis, and/or a decrease or eradication of tumor growth.

The term “efficacy” of a treatment or method according to the invention can be measured based on changes in the course of disease or condition in response to a use or a method according to the invention. For example, in the case of an inflammatory skin disorder like psoriasis, the efficacy of a treatment or method according to the invention can be measured by a reduction of patches, papules or plaques, and desquamations, on the skin, a reduction of the crisis frequency, a reduction of scratching, a reduction in the psoriasis area or/and of the Severity Index (PASI), a reduction of inflammatory, proliferation, and other specific markers (e.g. I16, PTGS2 mRNA and or proteins). For example, in the case of skin cancers, the efficacy of a treatment or method according to the invention regarding can be measured by a reduction of tumor volume, and/or an increase of progression free survival time, and/or a decreased risk of relapse post-resection for primary cancer.

The term “effective amount” as used herein refers to an amount of at least one miRNA inhibitor useful in the invention, composition or pharmaceutical formulation thereof, that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of miRNA inhibitor sufficient to reduce the progression of the disease, notably to reduce or inhibit the symptoms of the skin disorder and thereby elicit the response being sought (i.e. an “inhibition effective amount”).

### Agents and Compositions

### MiR-21-3p Inhibitors

In a first aspect, the invention provides inhibitors of miR-21-3p, in particular inhibitors of miR-21-3p for use as a medicament and the use of inhibitors of miR-21-3p for the preparation of a medicament, as well as compositions comprising said inhibitors.

An inhibitor of miR-21-3p is a compound which reduces or represses the activity and/or expression of miR-21-3p. In a particular embodiment, inhibitors of miR-21-3p according to the invention reduces or represses the activity and/or expression of miR-21-3p by interacting with the mature miR-21-3p, the pri-miR-21 and/or the pre-miR-21. Preferably, the inhibitor has no effect or substantially no effect on miRNAs which are not miR-21-3p.

According to the invention, inhibitors of miR-21-3p include polypeptides, nucleic acids (e.g. antisense oligonucleotides, shRNA, siRNA, antagomirs), peptide nucleic acids (PNA), or small chemical molecules, which are able to repress miR-21-3p expression and/or inhibit miR-21-3p activity.

It is understood that the variants and fragments of nucleic acids as described herewith, which constitute the miR-21-3-p inhibitors according to the invention, are able to reduce or repress the activity and/or expression of miR-21-3p.

MiR-21-3p expression can be determined by any standard method known in the art, including quantitative PCR, RT-PCR, real-time PCR, RT-LAMP, RNA sequencing, bead-based flow cytometry, microarrays, Northern blotting, dot blotting, RNase protection assays, primer extension analysis, miRNA in situ hybridization, and Invader™ assays.

MiR-21-3p activity can be evaluated by determining the binding of miR-21-3p to at least one of its target(s), the assessment of de-repression of at least one of its target(s), for instance, by quantitative PCR, Western blot analysis and genome-wide transcriptional (e.g. microarray, RNA sequencing) or proteomic analyses, and miRNA reporter assays.

In one embodiment, appropriate inhibitors of miR-21-3p correspond to oligonucleotides hybridizing to:
(i) the nucleic acid sequence of miR-21-3p or a fragment thereof, in particular human miR-21-3p of SEQ ID NO: 1 or murine miR-21-3p of SEQ ID NO: 2 or a variant thereof or a fragment thereof, and/or
(ii) the seed region of miR-21-3p, such as a sequence corresponding to nucleotides 2 to 7 of SEQ ID NO: 1 or nucleotides 2 to 7 of SEQ ID NO: 2 or a variant thereof, and/or
(iii) a fragment of at least 10 contiguous nucleotides from the nucleic acid sequence of miR-21-3p, such as human miR-21-3p of SEQ ID NO: 1 or murine miR-21-3p of SEQ ID NO: 2 or a variant thereof, optionally comprising the seed region of miR-21-3p, such as a sequence corresponding to nucleotides 2 to 7 of SEQ ID NO: 1 or nucleotides 2 to 7 of SEQ ID NO: 2 or a variant thereof.

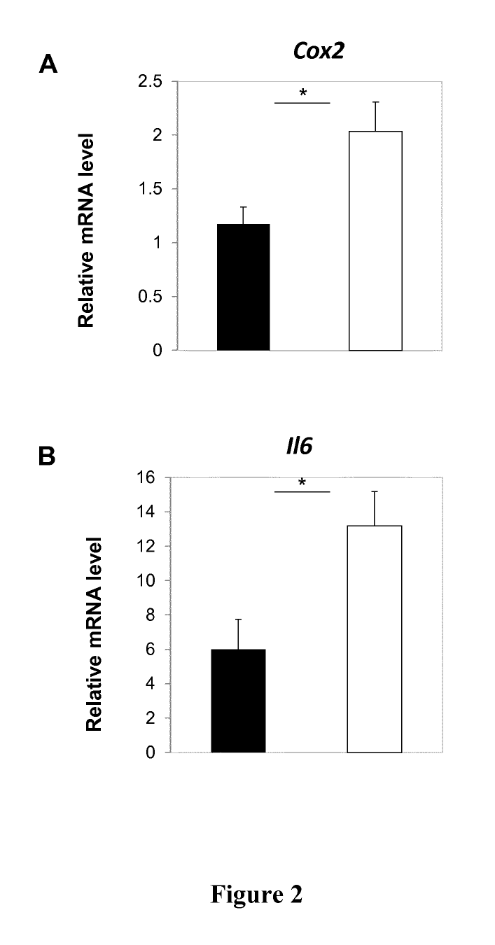

It is believed that the seed region of mammalian miR-21-3p extends from nucleotides 2 to 7 of mature miR-21-3p sequence (FIG. 2 of Lo et al., 2013, Plos One, 8(9):1-11). For instance, the seed region of human miR-21-3p could correspond to nucleotides 2 to 7 of SEQ ID NO: 1, i.e. AACACC, the seed region of murine miR-21-3p could correspond to nucleotides 2 to 7 of SEQ ID NO: 2, i.e. AACAGC.

In another embodiment, appropriate inhibitors of miR-21-3p correspond to oligonucleotides hybridizing to:

(i) the nucleic acid sequence of miR-21-3p or a fragment thereof, in particular human miR-21-3p of SEQ ID NO: 1 or murine miR-21-3p of SEQ ID NO: 2 or a variant thereof or a fragment thereof, and/or
(ii) the seed region of miR-21-3p, such as a sequence corresponding to nucleotides 2 to 8 of SEQ ID NO: 1 or nucleotides 2 to 8 of SEQ ID NO: 2 or a variant thereof, and/or
(iii) a fragment of at least 10 contiguous nucleotides from the nucleic acid sequence of miR-21-3p, such as human miR-21-3p of SEQ ID NO: 1 or murine miR-21-3p of SEQ ID NO: 2 or a variant thereof, optionally comprising the seed region of miR-21-3p, such as a sequence corresponding to nucleotides 2 to 8 of SEQ ID NO: 1 or nucleotides 2 to 8 of SEQ ID NO: 2 or a variant thereof,
and/or
(iv) the extended seed region of miR-21-3p, such as a sequence corresponding to nucleotides 2 to 16 of SEQ ID NO: 1 or nucleotides 2 to 16 of SEQ ID NO: 2 or a variant thereof.

In a particular embodiment, the inhibitor of miR-21-3p according to the invention corresponds to an oligonucleotide hybridizing to a variant of the specified nucleic acid sequence, wherein said variant differs from said nucleic acid sequence by one nucleotide.

In a particular embodiment, the inhibitor of miR-21-3p according to the invention corresponds to an oligonucleotide hybridizing to a variant of the specified nucleic acid sequence, wherein said variant differs from said nucleic acid sequence by two nucleotides.

In a particular embodiment, the inhibitor of miR-21-3p according to the invention corresponds to an oligonucleotide hybridizing to a variant of the specified nucleic acid sequence, wherein said variant differs from said nucleic acid sequence by one nucleotide.

Typically, a fragment of the nucleic acid sequence of miR-21-3p that hybridizes with the miR-21-3p inhibitors according to the invention may have about 5 to 21 nucleotides, in particular about 5, 6, 7, 8, 9 or 10 to 21 nucleotides, more particularly about 20 nucleotides e.g. 15-21 nucleotides, for example about 15, 16, 17, 18, 19, 20, or 21 nucleotides.

Appropriate miR-21-3p inhibitors may include single or double stranded oligonucleotides which are able to bind to mature miR-21-3p, in particular to the seed region of mature miR-21-3p, or to the passenger strand of miR-21 precursors, and inhibit the activity of mature miR-21-3p, prevent or inhibit its production and/or increase its rate of depletion.

Appropriate oligonucleotides may be oligodeoxyribonucleotides, oligoribonucleotides or modified oligonucleotides as described below.

In one embodiment of the invention, an inhibitor of miR-21-3p is a single stranded oligonucleotide which has a sequence which is sufficiently complementary to the sequence of miR-21-3p to hybridize to said miR-21-3p by Watson-Crick base-pairing.

In a particular embodiment, an inhibitor of miR-21-3p comprises a single stranded oligonucleotide complementary to miR-21-3p nucleic acid sequence and/or complementary to a fragment of miR-21-3p, said fragment reducing or repressing the activity and/or expression of miR-21-3p.

In a particular embodiment, an inhibitor of miR-21-3p comprises a single stranded oligonucleotide complementary to miR-21-3p nucleic acid sequence and/or complementary to a fragment of miR-21-3p, said fragment comprising the seed region of miR-21-3p.

In another particular embodiment, said fragment comprises at least 6, 7, 8, 9, or at least 10 nucleotides.

In another embodiment, an inhibitor of miR-21-3p comprises a single stranded oligonucleotide, hybridizing to:
(i) the nucleic acid sequence of miR-21-3p or a fragment thereof, such as human miR-21-3p of SEQ ID NO: 1 or murine miR-21-3p of SEQ ID NO: 2 or a variant thereof or a fragment thereof, and/or
(ii) the seed region of miR-21-3p, in particular a sequence corresponding to nucleotides 2 to 7 of SEQ ID NO: 1 or nucleotides 2 to 7 of SEQ ID NO: 2 or a variant thereof, and/or
(iii) a fragment of at least 10 contiguous nucleotides from the nucleic acid sequence of miR-21-3p, in particular human miR-21-3p of SEQ ID NO: 1 or murine miR-21-3p of SEQ ID NO: 2 or a variant thereof, optionally comprising the seed region of miR-21-3p, in particular a sequence corresponding to nucleotides 2 to 7 of SEQ ID NO: 1 or nucleotides 2 to 7 of SEQ ID NO: 2 or a variant thereof.

In a further embodiment, an inhibitor of miR-21-3p is an oligonucleotide, in particular a single stranded oligonucleotide, comprising SEQ ID NO: 3, or a variant thereof, or a fragment thereof able to reduce or repress the activity and/or expression of miR-21-3p.

In a further embodiment, an inhibitor of miR-21-3p is an oligonucleotide, in particular a single stranded oligonucleotide, comprising SEQ ID NO: 3, or a variant thereof, or a fragment thereof comprising nucleotides 15 to 20 of SEQ ID NO: 3.

In a particular embodiment, said variant of the nucleic acid sequence specified herewith differs from said nucleic acid sequence by one nucleotide.

In a particular embodiment, said variant of the nucleic acid sequence specified herewith differs from said nucleic acid sequence by two nucleotides.

In a more particular embodiment, said fragment comprises at least 6, 7, 8, 9, 10, 11, 12, 13, 14, 15, 16, 17, 18, 19, or 20 contiguous nucleotides from SEQ ID NO: 3.

In a particular embodiment, an inhibitor of miR-21-3p is an oligonucleotide, in particular a single stranded oligonucleotide, comprising a fragment of at least 10 contiguous nucleotides from SEQ ID NO: 3.

In a particular embodiment, an inhibitor of miR-21-3p is an oligonucleotide, in particular a single stranded oligonucleotide, comprising a fragment of at least 10 contiguous nucleotides from SEQ ID NO: 3, or a variant thereof, and comprising nucleotides 15 to 20 of SEQ ID NO: 3.

In a further particular embodiment, an inhibitor of miR-21-3p is an oligonucleotide, in particular a single stranded oligonucleotide, comprising SEQ ID NO: 4 or SEQ ID NO: 5.

In a further embodiment, an inhibitor of miR-21-3p is an oligonucleotide, in particular a single stranded oligonucleotide, comprising SEQ ID NO: 6, or a variant thereof, or a fragment thereof able to reduce or repress the activity and/or expression of miR-21-3p.

In a further embodiment, an inhibitor of miR-21-3p is an oligonucleotide, in particular a single stranded oligonucleotide, comprising SEQ ID NO: 6, or a variant thereof, or a fragment thereof comprising nucleotides 15 to 20 of SEQ ID NO: 6.

In a more particular embodiment, said fragment comprises at least 6, 7, 8, 9, 10, 11, 12, 13, 14, 15, 16, 17, 18, 19, or 20 contiguous nucleotides from SEQ ID NO: 6.

In a particular embodiment, an inhibitor of miR-21-3p is an oligonucleotide, in particular a single stranded oligonucleotide, comprising a fragment of at least 10 contiguous nucleotides from SEQ ID NO: 6, or a variant thereof.

In a particular embodiment, an inhibitor of miR-21-3p is an oligonucleotide, in particular a single stranded oligonucleotide, comprising a fragment of at least 10 contiguous nucleotides from SEQ ID NO: 6, or a variant thereof, and comprising nucleotides 15 to 20 of SEQ ID NO: 6.

In a further particular embodiment, an inhibitor of miR-21-3p is an oligonucleotide, in particular a single stranded oligonucleotide, comprising SEQ ID NO: 7 or SEQ ID NO: 8.

In a particular embodiment of the invention, a variant of the nucleic acid sequence specified herewith differs from said nucleic acid sequence by one nucleotide.

In a particular embodiment of the invention, a variant of the nucleic acid sequence specified herewith differs from said nucleic acid sequence by two nucleotides.

Typically, an appropriate oligonucleotide for inhibition of miR-21-3p may have about 6 to 30 nucleotides, in particular about 10 to 30 nucleotides, more particularly about 20 nucleotides e.g. 15-21 nucleotides, for example about 15, 16, 17, 18, 19, 20, or 21 nucleotides.

According to a particular embodiment, is provided a miR-21-3p inhibitor for inhibition of miR-21-3p having about 5 to 30 nucleotides.

In a further particular embodiment, is provided a miR-21-3p inhibitor having a oligonucleotide sequence consisting in any one of:
(i) a fragment of SEQ ID NO: 3 or a variant thereof,
(ii) SEQ ID NO: 4 or SEQ ID NO: 5 or a variant thereof, or a fragment thereof,
(iii) SEQ ID NO: 6, or a variant thereof, or a fragment thereof optionally comprising nucleotides 15 to 20 of SEQ ID NO: 6,
(iv) a fragment of SEQ ID NO: 6 optionally comprising nucleotides 15 to 20 of SEQ ID NO: 6,
(v) SEQ ID NO: 7 or SEQ ID NO: 8,
(vi) SEQ ID NO: 29, or a variant thereof, or a fragment thereof.

According to a particular embodiment, a miR-21-3p inhibitor has a oligonucleotide sequence of the invention further modified with a LNA moiety, i.e. is a LNA-modified oligonucleotide.

As will be understood by one skilled in the art, when the inhibitor of miR-21-3p is an RNA molecule, said inhibitor may comprise any one of the above-mentioned sequences, except that the five-carbon pentose of the backbone is a ribose instead of a deoxyribose, and the nitrogenous base uracil (U) replaces the thymine (T).

In another embodiment, the inhibitor of miR-21-3p corresponds to a double stranded DNA placed under the control of a promoter that allows expression of a transcript complementary to miR-21-3p, or complementary to a fragment of miR-21-3p optionally comprising the miR-21-3p seed region, as for example in a sponge vector. Thus, the sequence comprised in the sponge vector can be identical to the seed region of miR-21-3p seed region except for a few (e.g. about 1, 2, 3) different nucleotides. The exact number of different nucleotides are determined empirically for each miRNA, by the skilled person.

In particular embodiments, the oligonucleotides for the inhibition of miR-21-3p activity are chemically modified.

The chemical modifications of said oligonucleotides may comprise modifications to the nucleobases, the backbone residues, and/or the internucleoside linkers of said oligonucleotides.

Modifications to one or more nucleobases of said oligonucleotides may comprise one or more alkylated purines and pyrimidines, acylated purines and pyrimidines, and other heterocycles. These classes of pyrimidines and purines are known in the art and include pseudoisocytosine, N4,N4-ethanocytosine, 8-hydroxy-N-6-methyladenine, 4-acetylcytosine, 5-(carboxyhydroxylmethyl)uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N-6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine and 1-methylcytosine.

Modifications to one or more backbone residues of said oligonucleotides may comprise one or more of the following: 2′ sugar modifications such as 2′-O-methyl (2′-OMe), 2′-O-methoxyethyl (2′-MOE), 2′-O-methoxyethoxy, 2′-Fluoro (2′-F), 2′-Allyl, 2′-O-[2-(methylamino)-2-oxoethyl], 2′-O—(N-methylcarbamate); 4′ sugar modifications including 4′-thio, 4′-CH₂—O-2′-bridge, 4-(CH₂)₂—O-2′-bridge; Locked Nucleic Acid (LNA); Peptide Nucleic Acid (PNA); Intercalating nucleic acid (INA); Twisted intercalating nucleic acid (TINA); Hexitol nucleic acids (HNA); arabinonucleic acid (ANA); cyclohexane nucleic acids (CNA); cyclohexenylnucleic acid (CeNA); threosyl nucleic acid (TNA); Morpholino oligonucleotides; Gap-mers; Mix-mers; Incorporation Arginine-rich peptides; addition of 5′-phosphate to synthetic RNAs; RNA Aptamers; or any combinations thereof.

Modifications to one or more internucleoside linkers of said oligonucleotides may comprise one or more of the following: Phosphorothioate, phosphoramidate, phosphorodiamidate, phosphorodithioate, phosphoroselenoate, phosphorodiselenoate, phosphoroanilothioate and phosphoranilidate, or any combinations thereof.

Locked Nucleic Acid (LNA) nucleosides are a class of nucleic acid analogues in which the ribose ring is “locked” by a methylene bridge connecting the 2′-O atom and the 4′-C atom. LNA nucleosides contain the common nucleobases that appear in DNA and RNA and are able to form base pairs according to standard Watson-Crick base pairing rules. However, by “locking” the molecule with the methylene bridge the LNA is constrained in the ideal conformation for Watson-Crick binding. When LNA nucleosides are incorporated into a DNA or RNA oligonucleotide, this makes the pairing with a complementary nucleotide strand more rapid and increases the stability of the resulting duplex. This increased stability, as well as the high hybridization affinity and improved mismatch discrimination abilities, make LNA-modified oligonucleotides extremely potent antisense inhibitors, both for in vitro and in vivo use. When transfected in the cells, LNA-modified oligonucleotides display high nuclease resistance and low cytotoxicity. It is also possible to further modify LNA-modified oligonucleotides to include a phosphorothioate backbone, which further increases nuclease resistance of the oligonucleotide and improves the efficiency of the inhibition.

An LNA-modified olignonucleotide contains one or more units of an LNA monomer, preferably one or more 2′-O, 4′-C-methylene bridge monomers (oxy-LNA). An LNA-modified oligonucleotide however also may contain other LNA units in addition to or in place of an oxy-LNA group. In particular, preferred additional LNA units include 2′-thio-LNA (thio-LNA), 2′-HN-LNA (amino-LNA), and 2′-N(R)-LNA (amino-R-LNA)) monomers in either the D-B or L-a configurations or combinations thereof.

An LNA-modified oligonucleotide also may have other internucleoside linkages than the native phosphodiester, e.g. phosphoromonothioate, phosphorodithioate, and methylphosphonate linkages. The LNA-modified oligonucleotide can be fully modified with LNA (i.e. each nucleotide is an LNA unit), but it is generally preferred that the LNA-modified oligomers will contain other residues such as native DNA monomers, phosphoromonothioate monomers, methylphosphonate monomers or analogs thereof. In general, an LNA-modified oligonucleotide will contain at least about 5, 10, 15 or 20 percent LNA units, based on total nucleotides of the oligonucleotide, more typically at least about 20, 25, 30, 40, 50, 60, 70, 80 or 90 percent LNA units, based on total bases of the oligonucleotide.

In a particular embodiment, the inhibitor of miR-21-3p is a LNA-modified oligonucleotide.

In a still further embodiment, the inhibitor of miR-21-3p is a modified LNA-oligonucleotide hybridizing to:
(i) the nucleic acid sequence of miR-21-3p or a fragment thereof, in particular human miR-21-3p of SEQ ID NO: 1 or murine miR-21-3p of SEQ ID NO: 2 or a variant thereof, or a fragment thereof, and/or
(ii) the seed region of miR-21-3p, in particular a sequence corresponding to nucleotides 2 to 7 of SEQ ID NO: 1 or nucleotides 2 to 7 of SEQ ID NO: 2 or a variant thereof, and/or
(iii) a fragment of at least 10 contiguous nucleotides from the nucleic acid sequence of miR-21-3p, in particular human miR-21-3p of SEQ ID NO: 1 or murine miR-21-3p of SEQ ID NO: 2 or a variant thereof, optionally comprising the seed region of miR-21-3p, such as a sequence corresponding to nucleotides 2 to 7 of SEQ ID NO: 1 or nucleotides 2 to 7 of SEQ ID NO: 2 or a variant thereof,
wherein the ribose ring of one or more nucleotides of said oligonucleotide is locked by a methylene bridge connecting the 2′-O atom and the 4′-C atom.

In a more particular embodiment, the inhibitor of miR-21-3p is a LNA-modified oligonucleotide comprising SEQ ID NO: 3, or a variant thereof, or a fragment thereof, wherein the ribose ring of one or more nucleotides of said oligonucleotide is locked by a methylene bridge connecting the 2′-O atom and the 4′-C atom.

In a more particular embodiment, the inhibitor of miR-21-3p is a LNA-modified oligonucleotide comprising SEQ ID NO: 3, or a variant thereof, or a fragment thereof comprising nucleotides 15 to 20 of SEQ ID NO: 3, wherein the ribose ring of one or more nucleotides of said oligonucleotide is locked by a methylene bridge connecting the 2′-O atom and the 4′-C atom.

Still more particularly, said fragment comprises at least 6, 7, 8, 9, 10, 11, 12, 13, 14, 15, 16, 17, 18, 19, or 20 contiguous nucleotides from SEQ ID NO: 3.

In a particular embodiment, the inhibitor of miR-21-3p is a LNA-modified oligonucleotide comprising a fragment of at least 10 contiguous nucleotides from SEQ ID NO: 3, or a variant thereof.

In a particular embodiment, the inhibitor of miR-21-3p is a LNA-modified oligonucleotide comprising a fragment of at least 10 contiguous nucleotides from SEQ ID NO: 3, or a variant thereof, comprising nucleotides 15 to 20 of SEQ ID NO: 3, wherein the ribose ring of one or more nucleotides of said oligonucleotide is locked by a methylene bridge connecting the 2′-O atom and the 4′-C atom.

In a still more particular embodiment, the inhibitor of miR-21-3p is a LNA-modified oligonucleotide comprising SEQ ID NO: 4 or SEQ ID NO: 5, wherein the ribose ring of one or more nucleotides of said oligonucleotide is locked by a methylene bridge connecting the 2′-O atom and the 4′-C atom.

In a further embodiment, an inhibitor of miR-21-3p is a LNA-modified oligonucleotide comprising SEQ ID NO: 6, or a variant thereof, or a fragment thereof, wherein the ribose ring of one or more nucleotides of said oligonucleotide is locked by a methylene bridge connecting the 2′-O atom and the 4′-C atom.

In a further embodiment, an inhibitor of miR-21-3p is a LNA-modified oligonucleotide comprising SEQ ID NO: 6, or a variant thereof, or a fragment thereof comprising nucleotides 15 to 20 of SEQ ID NO: 6, wherein the ribose ring of one or more nucleotides of said oligonucleotide is locked by a methylene bridge connecting the 2′-O atom and the 4′-C atom.

More particularly, said fragment comprises at least 6, 7, 8, 9, 10, 11, 12, 13, 14, 15, 16, 17, 18, 19, or 20 contiguous nucleotides from SEQ ID NO: 6.

In a particular embodiment, the inhibitor of miR-21-3p is a LNA-modified oligonucleotide comprising a fragment of at least 10 contiguous nucleotides from SEQ ID NO: 6, or a variant thereof, wherein the ribose ring of one or more nucleotides of said oligonucleotide is locked by a methylene bridge connecting the 2′-O atom and the 4′-C atom.

In a particular embodiment, the inhibitor of miR-21-3p is a LNA-modified oligonucleotide comprising a fragment of at least 10 contiguous nucleotides from SEQ ID NO: 6, or a variant thereof, comprising nucleotides 15 to 20 of SEQ ID NO: 6, wherein the ribose ring of one or more nucleotides of said oligonucleotide is locked by a methylene bridge connecting the 2′-O atom and the 4′-C atom.

In a further embodiment, an inhibitor of miR-21-3p is a LNA-modified oligonucleotide comprising SEQ ID NO: 7 or SEQ ID NO: 8, wherein the ribose ring of one or more nucleotides of said oligonucleotide is locked by a methylene bridge connecting the 2′-O atom and the 4′-C atom.

In another particular embodiment, the inhibitor of miR-21-3p is an antagomir or a chemically modified antagomir including, for instance, 2′ methoxi groups and phosphothioates. Production of antagomirs may be achieved for instance as disclosed in Krutzfeldt et al., 2005, Nature, 438, 685-689).

Modified oligonucleotides may also contain one or more sugar mimetics instead of a pentofuranosyl sugar. Suitable sugar mimetics include cyclobutyl moieties, azido-ribose, carbocyclic sugar analogues a-anomeric sugars; epimeric sugars such as arabinose, xyloses or lyxoses, pyranose sugars, furanose sugars, and sedoheptulose.

In some embodiments, both the sugar and the backbone linkage of one or more, preferably all of the nucleotides in a modified oligonucleotide may be replaced with non-natural groups. The bases are maintained for hybridization with miR-21-3p. Suitable modified oligonucleotides may include peptide nucleic acids (PNA). In PNA, the oligonucleotide sugar-backbone is replaced with an amide containing backbone, in particular an aminoethylglycine backbone. The bases are retained and are bound directly or indirectly to aza-nitrogen atoms of the amide portion of the backbone.

In a further embodiment, modified oligonucleotides may be chemically linked to one or more moieties or groups which enhance the activity, cellular distribution or cellular uptake of the oligonucleotide. Suitable moieties include lipid moieties such as cholesterol, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. In a particular embodiment, said oligonucleotides and/or modified oligonucleotides are chemically linked to cholesterol.

In another embodiment, the inhibitor of miR-21-3p is a polypeptide or a peptide that is able to repress said micro-RNA expression and/or inhibit said micro-RNA function/activity.

In another embodiment, the inhibitor of miR-21-3p is a peptide nucleic acid (PNA) that is able to repress said micro-RNA expression and/or inhibit said micro-RNA function/activity.

In another embodiment, the inhibitor of miR-21-3p is a small chemical molecule that is able to repress said micro-RNA expression and/or inhibit said micro-RNA function/activity.

In a still other embodiment, the inhibitor of miR-21-3p activity is a RNA-binding protein or a target protector (TP) morpholinos/a miRNA target protector (can be designed as described in Staton et al., 201 1, Nature Protocols, 6(12): 2035-2049).

In a particular embodiment, said RNA-binding protein or target protector (TP) morpholinos is able to inhibit the ability of miR-21-3p to bind to Mmp1 mRNA (Matrix metalloproteinase-1) or Smad7 mRNA (SMAD family member 7).

RNA-binding proteins useful in the invention can be those described in van Kouwenhove et al., 2011, supra). Target protector (TP) morpholinos useful in the invention can be designed as described in Staton et al., 2011, Nature Protocols, 6(12): 2035-2049).

### Compositions

In another embodiment, the invention relates to a composition comprising a miR-21-3p inhibitor as described above or a vector comprising a nucleic acid encoding said miR-21-3p inhibitor.

In a particular embodiment, it is provided a cosmetic composition comprising a miR-21-3p inhibitor as described above or a vector comprising a nucleic acid encoding said miR-21-3p inhibitor.

In an alternative embodiment, it is provided the use of said cosmetic composition for improving skin appearance and/or skin comfort, e.g. for increasing or restoring skin suppleness, smoothness, and/or improving or restoring skin homeostasis.

In another embodiment, the invention relates to a pharmaceutical composition comprising a miR-21-3p inhibitor as described above or a vector comprising a nucleic acid encoding said miR-21-3p inhibitor, and a pharmaceutically acceptable carrier.

The invention provides pharmaceutical or therapeutic agents as compositions and methods for treating a subject, preferably a mammalian subject, and most preferably a human patient who is suffering from a medical disorder, in particular a skin disorder, in particular a skin disorder selected from inflammatory skin disorders such as psoriasis, dermatitis such as ectopic dermatitis, acne, rosacea, Stevens-Johnson syndrome, toxic epidermal necrolysis, systemic lupus erythematosus, skin allergies, atopic eczema, parakeratosis, keratosis, skin cancers such as squamous cell carcinoma, basal cell carcinomas and melanomas, leprosy, vitiligo, epidermolytic ichthyosis, UV skin photodamages such as skin ageing following UV exposure, topical allergy, UV erythema, skin ageing, wounds, and non-pre-malignant or pre-malignant structure affecting the skin such as actinic keratosis and seborrheic keratosis.

Compositions or formulations according to the invention may be administered as a pharmaceutical formulation which can contain one or more agents according to the invention in any form described herein.

The compositions according to the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) use by injection or continuous infusion. Injectable compositions are typically based upon injectable sterile saline or phosphate-buffered saline or other injectable carriers known in the art. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Compositions of this invention may be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Preservatives include, but are not limited to, methyl or propyl p-hydroxybenzoate and sorbic acid. Dispersing or wetting agents include but are not limited to poly(ethylene glycol), glycerol, bovine serum albumin, Tween®, Span®.

Compositions of this invention may also be formulated as a depot preparation, which may be administered by implantation or by intramuscular injection.

Solid compositions of this invention may be in the form of tablets or lozenges formulated in a conventional manner.

The compounds of this invention can also be administered in sustained release forms or from sustained release drug delivery systems.

According to a particular embodiment, compositions according to the invention are for topical use.

In a particular embodiment, the topical compositions of the present invention may be applied to skin (preferentially the epidermis, but also dermis, hypodermis, hair fbllicles, etc.), mucous membrane (present in vagina, lung, lips, mouth, etc.) and other epithelium subject to inflammation due to their susceptibility to external injury such as urothelium, gastric epithelia or lung epithelia).

In a particular embodiment, the topical compositions of the present invention may also include one or more of the following: a skin penetration enhancer, an emollient, such as isopropyl myristate, petrolatum, silicones (e.g., methicone, dimethicone), oils, mineral oils, and fatty acid esters; a humectant, such as glycerin or caprylyl glycol, a skin plumper, such as palmitoyl oligopeptide, collagen, or collagen and/or glycosaminoglycan (GAG) enhancing agents, a sunscreen, such as avobenzone, an exfoliating agent, and an antioxidant. Suitable exfoliating agents include, for example, alpha-hydroxyacids, beta-hydroxyacids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. A convenient exfoliating agent is glycolic acid. When present, the exfoliating agent may comprise from about 0.1 wt % to about 80 wt % of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g. ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may comprise an antioxidant preferably from about 0.001 wt % to about 10 wt %, and more preferably from about 0.01 wt % to about 5 wt %, of the total weight of the composition.

Other conventional additives include: vitamins, such as tocopherol and ascorbic acid; vitamin derivatives such as ascorbyl monopalmitate; thickeners such as hydroxyalkyl cellulose; gelling agents; structuring agents, metal chelating agents such as EDTA; pigments; colorants, and pH adjusters. The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders.

Particularly in case of topical administration, the composition may be formulated in a variety of product forms, such as an emulsion, lotion, cream, serum, spray, aerosol, cake, ointment, essence, gel, paste, patch, pencil, towelette, mask, stick, foam, elixir, concentrate, and the like. In particular, the composition is formulated as an emulsion, lotion, cream, ointment, serum or gel.

In a further particular embodiment, the miR-21-3p inhibitor of the invention is comprised in a diphenylcyclopropenone (DPCP) gel formulation. An example of such DPCP gel formulation is described in Hagen et al. (2013, J Drugs Dermatol, 12(2): 152).

In a still further embodiment, the miR-21-3p inhibitor of the invention is comprised in a poloxamere based formulation.

In a particular embodiment, the miR-21-3p inhibitor of the invention is comprised in an alcohol or water based formulation.

In another particular aspect, the compositions according to the invention are adapted for delivery by repeated administration.

Further materials as well as formulation processing techniques and the like are set out in Part 5 of Remington's “The Science and Practice of Pharmacy”, 22^{nd} Edition, 2012, University of the Sciences in Philadelphia, Lippincott Williams & Wilkins, which is incorporated herein by reference.

When the agents and compositions according to the invention comprise nucleic acids as described herewith, those can be prepared by incorporating the nucleic acid as an active ingredient together with a base which is commonly used for an agent for gene therapy.

When nucleic acids are incorporated into a virus vector, virus particles containing recombinant vectors are prepared, and the resultants are incorporated with a base which is commonly used for an agent for gene therapy.

As a base used for incorporating the nucleic acid as an active ingredient, a base that is commonly used for an injection can be used. Examples include distilled water, a salt solution of sodium chloride or a mixture of sodium chloride and an inorganic salt, a solution of mannitol, lactose, dextran, or glucose, an amino acid solution of glycine or arginine, an organic acid solution, and a mixed solution of a salt solution and a glucose solution. Alternatively, an adjuvant, such as a regulator of osmotic pressure, a pH adjuster, vegetable oil, or a surfactant, may be added to the base in accordance with a technique known in the art to prepare an injection in the form of a solution, suspension, or dispersion. Such injection can be prepared in the form of a preparation to-be-dissolved before use via operations, such as pulverization or lyophilization.

Formulations for topical administration of cosmetic compositions comprising the miR-21-3p inhibitors according to the invention can include lipid, water or alcohol base formulations like cream or shampoo.

In a particular embodiment, the topical composition according to the invention includes a humectant such as glycerol, an organosulfur compound such as dimethyl sulfoxide (DMSO) (formula (CH₃)₂SO) and a transfection agent such as the in vivo-jetPEI® from Polyplus-transfection® (such as described in U.S. Pat. No. 6,013,240, EP No. 0770140). In a still further embodiment, the topical composition is a formulation of a miR-21-3p inhibitor-containing lotion appropriate for topical application on human skin. Said lotion according to the invention may comprise glycerol as humectant, DMSO, a transfection reagent (RNAimax, Invitrogen, catalogue number 13778030) and 50 uM of miR-21-3p LNA-inhibitor diluted in PBS (Exiqon, I-hsa-miR-21-3p product number 500150, as described in Example 11) for example at the following respective volume proportions: 9:1:1:8. 16 μl of lotion were used by application to each 0.5 mm² biopsies. See in material and method (MiR-21-3p inhibitor topical lotion formulation). In a particular embodiment, the composition according to the invention is under the form of a cream, ointment, lotion or gel. Depending on the intended use (cosmetic/therapeutic use), the administrative mode, the efficiency and the toxicological parameters, the severity of the disorder as well as characteristics of the patient, the exact nature and proportions of the ingredients comprised in the composition of the invention may change. The formulation can include one or several co-agents such as corticosteroids.

### Uses and Methods According to the Invention in Therapy

In another aspect of the invention, it is provided a miR-21-3p inhibitor as described herewith for use in the prevention and/or treatment of skin disorders, in particular inflammatory skin disorders, keratosis, and skin cancers.

In an alternative aspect of the invention is provided the use of a miR-21-3p inhibitor as described herewith for the manufacture of a medicament for the prevention and/or treatment of skin disorders, in particular inflammatory skin disorders, keratosis, and skin cancers.

In a still alternative aspect of the invention is provides a method of preventing and/or treating skin disorders, in particular inflammatory skin disorders, keratosis, and skin cancers, said method comprising administering in a subject in need thereof a miR-21-3p inhibitor as described herewith, or a pharmaceutical formulation thereof.

In a particular embodiment of the above-mentioned aspects of the invention, said skin disorder is selected from inflammatory skin disorders (including psoriasis, dermatitis, such as atopic dermatitis, acne, rosacea, Stevens-Johnson syndrome, toxic epidermal necrolysis, systemic lupus erythematosus, skin allergies, atopic eczema, parakeratosis), keratosis, skin cancers (including squamous cell carcinoma, basal cell carcinomas and melanomas), leprosy, vitiligo, epidermolytic ichthyosis, UV photodamages, topical allergy, UV erythema, skin ageing following UV exposure, wounds and non-pre-malignant or pre-malignant structure affecting the skin (such as actinic keratosis and seborrheic keratosis).

In a still more particular embodiment, said skin disorder is an inflammatory skin disorder, particularly an inflammatory skin disorder selected from psoriasis, dermatitis such as ectopic dermatitis, acne, rosacea, Stevens-Johnson syndrome, toxic epidermal necrolysis, systemic lupus erythematosus, skin allergies, atopic eczema, parakeratosis.

In a still more particular embodiment, said skin disorder is psoriasis or dermatitis, more particularly psoriasis.

In a further embodiment, it is provided a miR-21-3p inhibitor as described herewith for use in the treatment of local inflammation following injury or wounds such as bump or hematoma.

In a still more particular embodiment, said skin disorder is selected from keratosis or skin cancers.

In a still more particular embodiment, said skin cancer is selected among squamous cell carcinomas, basal cell carcinomas and melanomas, more particularly squamous cell carcinoma.

In a still more particular embodiment, said skin disorder is a non-pre-malignant or pre-malignant structure affecting skin such as actinic keratosis and seborrheic keratosis.

In a particular embodiment, it is provided a miR-21-3p inhibitor as described herewith for use in the prevention of skin disorders, in particular inflammatory skin disorders, more particularly for use in the prevention of inflammatory dermatosis, immunosuppression caused by solar radiation especially in the case of UV hyper-sensibility pathologies and UV-based phototherapy.

According to a particular aspect, is provided a method, a use of a formulation according to the invention wherein said miR-21-3p inhibitor comprises a nucleotide of SEQ ID NO: 3 or a fragment of SEQ ID NO: 3 optionally comprising nucleotides 15 to 20 of SEQ ID NO: 3.

### Mode of Administration

Compositions of this invention may be administered in any manner including intravenous injection, intra-arterial, intraperitoneal injection, subcutaneous injection, intramuscular, intra-thecal, oral route, cutaneous application, direct tissue perfusion during surgery or combinations thereof.

Considering the easy accessibility of skin, topical delivery could reveal particularly appropriate for the oligonucleotides-based treatment of disorders of this tissue, which would have the obvious advantage that doses of oligonucleotides according to the invention required for an effect are considerably lower. In addition, topical administration also allows a more focused delivery, avoiding any undesirable side-effect potentially associated with systemic delivery.

In a particular embodiment compositions of miR-21-3p inhibitors can be delivered topically to skin (preferentially the epidermis, but also dermis, hypodermis, hair follicles, etc.), mucous membrane (present in vagina, lung, lips, mouth, etc.) and other epithelium such as urothelium, gastric epithelia or lung epithelia. When the agents and compositions according to the invention comprise oligonucleotides or vectors as described herewith, as active ingredients, those can be administered via, for example, simple direct injection of the nucleic acid molecule, the use of carriers of genetic material (adenoviruses, lentiviral vectors, adeno associated viruses), injection of conjugates including liposomes (vesicles consisting of an aqueous compartment enclosed in a phospholipid bilayer with the drug typically entrapped in the center aqueous layer), lipoplexes (spontaneously formed with the interaction of cationic lipids and negatively charged nucleic acids, are preferably prepared immediately before use), complexes with cholesterol, peptides (e.g. signal peptide for targeting specific cells), polymers or antibodies, and chemically modified oligonucleotides. Another delivery strategy includes the use of atelocollagen (e.g. obtained from type I collagen of calf dermis by pepsin treatment) whose surface is positively charged and the molecule can bond electrostatically with negatively charged nucleic acid molecules.

Successful local siRNA delivery relevant for dermatology has been described in mice employing in vivo skin electroporation (Inoue et al., 2007, J Gene Med, 9:994-1001), which could also be employed for administration of the miR-21-3p inhibitors described herewith.

### Combination

According to the invention, the agents and compositions according to the invention, and pharmaceutical formulations thereof can be administered alone or in combination with a co-agent useful in the treatment of a skin disorder.

The invention encompasses the administration of an agent or composition according to the invention and pharmaceutical formulations thereof, wherein said agent or composition is administered to an individual prior to, simultaneously or sequentially with other therapeutic regimens or co-agents useful in the treatment of a skin disorder, in particular skin inflammatory disorders, keratosis, and skin cancers, in a therapeutically effective amount.

An agent or composition according to the invention, or the pharmaceutical formulation thereof, that is administered simultaneously with said co-agents can be administered in the same or different composition(s) and by the same or different route(s) of administration.

According to one embodiment, is provided a pharmaceutical formulation comprising an agent or composition according to the invention, combined with at least one co-agent useful in the treatment of a skin disorder, preferably inflammatory skin disorders, keratosis and skin cancers, and at least one pharmaceutically acceptable carrier.

Said co-agents useful in the treatment of a skin disorder include topical retinoids (e.g. tazarotene), calcineurin inhibitors (e.g. tacrolimus and pimecrolimus), salicylic acid, coal tar, phototherapy, topical corticosteroids, vitamin D analogues, anthralin, acitretin, methotrexate, ciclosporin, mycophenolate mofetil, interferon gamma, biological agents such as alefacept, etanercept, adalimumab, efalizumab and infliximab, phototherapy, chemophototherapy, for instance, for the treatment of inflammatory skin disorders like psoriasis.

Said co-agents useful in the treatment of a skin cancer include imiquimod, radiation therapy and topical chemotherapy.

### Subjects

In an embodiment, subjects according to the invention are subjects suffering from a skin disorder.

In a particular embodiment, subjects according to the invention are subjects suffering from inflammatory skin disorders, more particularly from psoriasis, dermatitis such as ectopic dermatitis, acne, rosacea, Stevens-Johnson syndrome, toxic epidermal necrolysis, systemic lupus erythematosus, skin allergies, atopic eczema and parakeratosis.

In another particular embodiment, subjects according to the invention are subjects suffering from keratosis or skin cancers, in particular selected from squamous cell carcinomas, basal cell carcinomas and melanomas, more particularly squamous cell carcinoma.

In a still other particular embodiment, subjects according to the invention are subjects suffering from leprosy, vitiligo, epidermolytic ichthyosis, UV photodamages, topical allergy, UV erythema, skin ageing such as skin ageing following UV exposure and wounds.

In a still other particular embodiment, subjects according to the invention are subjects suffering from inflammatory dermatosis, immunosuppression caused by solar radiation especially in the case of UV hyper-sensibility pathologies and UV-based phototherapy, relapsing event of psoriasis.

### Uses and Methods According to the Invention in Diagnostics

In another aspect of the invention is provided an ex-vivo method of prognosis and/or diagnosis of a skin disorder in a subject comprising determining, in a biological sample of said subject, the level of miR-21-3p.

In a particular embodiment, said ex-vivo method of prognosis and/or diagnosis of a skin disorder in a subject comprises:
a) Providing a biological sample from a subject;
b) Determining the level of miR-21-3p, in said sample;
c) Comparing the level of miR-21-3p determined in step b) with the level of miR-21-3p in a control sample:
wherein an increased level of miR-21-3p determined in step b) compared to the control sample is indicative of the subject being at risk for developing, or having, a skin disorder.

In the method mentioned herewith, the level of miR-21-3p can be determined by any method known in the art including, for example, quantitative PCR, RT-PCR, real-time PCR, RT-LAMP, RNA sequencing, bead-based flow cytometry, microarrays, Northern blotting, dot blotting, RNase protection assays, primer extension analysis, miRNA in situ hybridization, and Invader™ assays.

In the ex-vivo method of prognosis and/or diagnosis of a skin disease according to the invention, the biological sample is a skin sample. Said biological sample can comprise cells taken from a lesion or other site on the skin of the subject.

Appropriate control samples include cells from healthy (i.e. non-lesional) skin which is not affected by a skin disorder or skin affected with the same disorder but with an inferior severity degree. Control cells may also be obtained from a different subject than the one from whom the biological sample has been extracted and who is submitted to the test, for example a healthy individual not suffering from, or susceptible to, a skin disorder or suffering of the same disorder but with an inferior severity degree than the tested subject.

References cited herein are hereby incorporated by reference in their entirety. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

The invention having been described, the following examples are presented by way of illustration, and not limitation.

### EXAMPLES

The following abbreviations refer respectively to the definitions below:

3′UTR MMP1-Luc (Luciferase signal related to MMP1 3′UTR); Ac-UV (acute Ultra Violet); Angptl4 (angiopoietin-like 4); Chr-UV (Chronic Ultra Violet); Cox2 (cyclooxygenase 2); Ccl5 ((C—C motif) chemokine 5); Cdkn1A (also p21) (cyclin-dependent kinase inhibitor 1); Cdkn2B (also p15) (cyclin-dependent kinase inhibitor 2); Cxcl10 (C—X—C motif chemokine 10); HaCaT (immortal human keratinocyte cell line); IL1α (interleukin 1α); IL1β (interleukin 1β); IL1RAP (interleukin-1 receptor accessory protein); IL6 (interleukin 6); MM (malignant melanoma); MMP1 (matrix metalloproteinase-1); Mut-MMP1-Luc (Luciferase signal related to mutated MMP1 3′UTR); NHM (normal melanocytes); NS (normal skin); p15 (cyclin-dependent kinase inhibitor 2B); p21 (cyclin-dependent kinase inhibitor 1); PM (primary melanoma); PPARβ/δ (peroxisome proliferator-activated receptor type β/δ); SCC (squamous cell carcinomas); Tgfβ-1 (transforming growth factor beta 1); Tnfα (tumor necrosis factor α); TS (tape stripping skin); UV (ultraviolet).

### Materials and Methods

Animal Model, Acute and Chronic UV Irradiation, GSK0660 Treatments

Hairless PPARβ/δ+/+ and PPARβ/δ−/− mice (Leibowitz et al. 2000, FEBS Lett. 473, 333-336) were housed in quarters with 12/12 h light/dark cycle and maintained with water and food ad libitum. UV irradiation of at least four mice per group was performed using a solar UV lamp (UV DUKE GL40E 40Watts, Griot SA, UVB/UVA<0.1%). For acute UV exposure, 9-12 weeks old females were irradiated on their backs, and UV radiation emission was controlled using a radiometer until a dose of 120 mJ/cm² of UVB was delivered. The animals were sacrificed and dorsal skin samples were harvested twenty-four hours after acute UV irradiation. For chronic treatment, animals were irradiated on their backs with a dose of 70 mJ/cm² of UVB as monitored using a radiometer, three times a week during a maximum period of 30 weeks. Dorsal skin free of any lesion was harvested after 12 weeks of UV exposure. Skin tumours larger than 5 mm of diameter were harvested after a maximum period of 30 weeks and diagnosed as papillomas or squamous cell carcinomas (SCC) by an expert pathologist. Non-irradiated aged-matched mice were used as controls. Samples were directly frozen in liquid nitrogen for RNA preparation or prepared for histological and in situ hybridization analysis. For GSK0660 treatment, 200 μl of GSK0660 (625 μg/μl in 70% ethanol; Sigma (™), G5797) was applied topically on the back 1 h prior to UV exposure.

All experiments involving animals described herein were approved by the Veterinary Office of the Canton Vaud (Switzerland) in accordance with the Federal Swiss Veterinary Office Guidelines and conform to the European Commission Directive 86/609/EEC.

MiR-21-3p Inhibitor In Vivo Sub-Cutaneous Injections

Mice received an injection of in vivo inhibitor “mmu-miR-21a-3p_inh., 5′-FAM labeled” (Exiqon Inhibitor Probe Number 199900, SEQ ID NO: 7: CCATCGACTGCTGTT) or in vivo inhibitor 3 mismatch control “mmu-miR-21a-3p_3MM, 5′-FAM labeled” as a control (SEQ ID NO: 9: CCCTAGACTGCTCTT) at a concentration of 20 mg/kg. Injections were performed 6 h prior and 30 minutes after acute UV irradiation under isoflurane anaesthesia. Mice were scarified 24 h after UV exposure. Sacrifice was immediately followed by the dermis/epidermis separation and sample snap-freezing in liquid nitrogen.

Extraction of Total RNA

Total RNA was isolated using TRIzol™ reagent (Invitrogen), according to the manufacturer protocol, and quantified using spectrophotometer at an optical density of 260 nm (Nanodrop ND1000). Quality of the RNA was assessed using a BioAnalyzer (Agilent).

Human Skin Biopsies

Cutaneous SCC samples were obtained anonymously from the Department of Dermatology, University Hospital of Lausanne, Switzerland. Normal skin was from healthy adult volunteers. Pathologists diagnosed SCC. Informed consent for research was obtained prior to routine diagnostic services.

Cell Culture, Transfection and Treatments

Human immortalized keratinocytes HaCaT cells were maintained in DMEM growth medium (Gibco) supplemented with 4500 mg/l glucose, 10% fetal bovine serum, 100 units/ml of penicillin G and 100 μg/ml of streptomycin. Cells were grown in a 5% CO₂ atmosphere at 37° C. For miR-21-3p functional analysis, cells were seeded and grown for 24 h, mimic (which amino acid sequence is identical to that of mature miR-21-3p, Miridian™, Thermo Scientific Dharmacon, cat number C-30123-01-0005) or control (amino acid sequence of cel-miR-67-3p of SEQ ID NO: 28 (UCACAACCUCCUAGAAAGAGUAGA, Miridian™, Thermo Scientific Dharmacon, cat number CN-001000-01-05) were transfected to the cells at a concentration of 50 nM using the Lipofectamine RNAi max (Invitrogen). Cells were harvested 24 h after transfection for gene expression analysis and 72 h after transfection for lipidome analysis.

mRNA Microarray Data Analysis

For hybridization, GeneChip MouseGene 1.0 ST arrays (Affymetrix Inc, Tokyo, Japan) were used (Genomic Technologies Facility-GTF, CIG) to analyze the expression a set of 28,000 gene-level probe sets (750,000 unique 25-mer oligonucleotide features). After hybridization and washing, the Affymetrix GeneChip Command Console software (AGCC) was used for array scanning. All these procedures were conducted according to the manufacturer's instructions. Affymetrix Quality Control software was employed for internal array normalization (default control probes were set as internal normalization controls). Background correction, inter-chips quantile normalization and summarization were manually processed using the “Robust Multi-Array Average” (“RMA”) R package. Data were ranked using the “Linear Models for Microarray Data” R package (“LIMMA”) for comparison of the different experimental conditions. In “LIMMA”, p-values were obtained from moderated t-statistics using Empirical Bayesian methods. P-values were then adjusted for multiple testing with Benjamini and Hochberg's method to control the false discovery rate. For mRNA expression arrays, probe sets showing a false discovery rate<0.05. Four technical replicates for each group (non-irradiated mice, 12 weeks and 30 weeks irradiated mice) were averaged for fbld change study. Statistical significance was set to p-value<0.001 by applying t-test analysis adjusted for multiple comparisons. Gene intensity changes higher than 1.5 were considered as biologically significant.

miRNA Microarray Data Analysis

Each RNA skin sample was prepared according to the Agilent's miRNA Microarray System protocol and loaded on the mouse microarray (Mouse miRNA Microarray Release 16.0) able to detect 567 mouse miRNAs (Sanger miRBase, release 10.1). Normalization procedures were based on the invariant procedure and the quantile normalization using the “normalize.quantiles” function from R package “affy” from the Bioconductor project (httpJ/www.bioconductor.org) as described in (Pradervand S 2009). The selected invariant probes had high mean expression and low standard deviation to reduce stochastic effects. Quantile normalization assumed that the global distribution of signal intensity did not change across arrays (n=4). After normalization, the <<LIMMA>> package was used to define a robust linear regression for inter-conditions fold change calculation. In “LIMMA”, p-values were obtained from moderated t-statistics using Empirical Bayesian methods. P-values were then adjusted for multiple testing with Benjamini and Hochberg's method to control the false discovery rate. Features showing a false discovery rate<0.1 were considered significant for miRNA arrays. Statistical significance was set to p-value<0.001 by applying t-test analysis adjusted for multiple comparisons. miRNA showing fold changes higher than 1.5 were selected.

Differences in major biological functions were identified using Ingenuity's pathway analysis (IPA) v2.0 (Ingenuity Systems, Redwood City, Calif.). The data files containing the probe identifier (gene accession numbers) and the corresponding changes in expression values (fold change (FC) and p-value) were uploaded into IPA. Pathways were considered significantly different when the right-tailed Fisher's exact test p-value threshold where below 0.005.

Reverse Transcription and Real-Time PCR

mRNA: one μg of total RNA was reverse transcribed with random hexamere primers (Promega) using the SuperScriptII Reverse Transcriptase (Invitrogen). Real-Time PCR was performed with SYBR Green PCR Master Mix (Applied Biosystems) using Stratagene Mx3000p thermo cycler. Primer sequences are listed below. Expression was related to the expression of the house keeping genes Gapdh and Eflal for mice samples and to Rp127 and Hprt for human samples and cells (GeNorm, M value<0.5). microRNA: Reverse-transcription was performed using the Exiqon miRCURY LNA™ Universal RT microRNA kit. Quantitative PCR was performed according to manufacturer protocol with microRNA LNA™ PCR primer sets and SYBR Green PCR Master Mix (Applied Biosystems). qPCR was performed on a MX3000P machine (Stratagene). miR-103 and sno234, for which the expression is stable in our model, were used as control for normalization.

Quantification of relative expression was based on the determination of the threshold cycle (Ct). Comparison of the relative expression is based on the two by two-sided Student's T-test for paired samples. Significance threshold was set at p-value<0.05.

mRNA Primer Sequences

| Name | Forward primer | Reverse primer |
|---|---|---|
| mmu-Gapdh | SEQ ID NO: 10: | SEQ ID NO: 11: |
| | GTATGACTCCACTACGGCAAA | TTCCCATTCTCGGCTTG |
| mmu-Ef1a1 | SEQ ID NO: 12: | SEQ ID NO: 13: |
| | CCTGGCAAGCCCATGTGT | TCATGTCACGAACAGCAAAGC |
| mmu-Pparβ/δ | SEQ ID NO: 14: | SEQ ID NO: 15: |
| | CGGCAGCCTCAACATGG | AGATCCGATCGCACTTCTCATAC |
| hsa-Pparβ/δ | SEQ ID NO: 16: | SEQ ID NO: 17: |
| | GCATGAAGCTGGAGTACGAGAAG | GCATCCGACCAAAACGGATA |
| mmu-Pparβ/δ | SEQ ID NO: 18: | SEQ ID NO: 19: |
| | CGGCAGCCTCAACATGG | AGATCCGATCGCACTTCTCATAC |
| hsa-P21 | SEQ ID NO: 20: | SEQ ID NO: 21: |
| | CTGTCACTGTCTTGTACCCT | GGTAGAAATCTGTCATGCTGG |
| hsa-P15 | SEQ ID NO: 22: | SEQ ID NO: 23: |
| | CGTGGGAAAGAAGGGAAGAGT | CCCCAGACGCGCAGC |

Hsa- and mmu-Angpt14, Tgfβ-1, hsa-IL6, hsa-COX2 (has-PTGS2), hsa-Mmp1, hsa-Rp127, hsa-Hprt have been purchased from Qiagen (QuantiTect primers).

miRNAs Primer Sequences

mmu/hsa-miR-21-3p; mmu/hsa-miR-21; mmu/hsa-miR-103 and a customized hsa- and mmu-pre-miR-21 primers have been purchased from Exiqon (microRNA LNA PCR primer sets).

hsa-pri-miR-21 forward primer: SEQ ID NO: 24:
TTTTGTTTTGCTTGGGAGGA
hsa-pri-miR-21 reverse primer: SEQ ID NO: 25:
AGCAGACAGTCAGGCAGGAT

miRNA Fluorescent In Situ Hybridization with Locked Nucleic Acid (LNA) Probes

Skin samples were fixed and cryopreserved at 4° C. in 4% in paraformaldehyde and 30% sucrose overnight. Samples were then frozen in optimum cutting temperature matrix (OCT) at −80° C., further cut in 10 μm sections (Cryostat, Leica), and mounted on slides. During the acetylation step, sections were washed in 0.1M of Triethanolamine/10 min, then Triethanolamine/0.25% acetic anhydride/10 min. 100 μl of Digoxigenin-labelled LNA probes (Exiqon) (25 nM in hybridization buffer) were added on each section and hybridization was performed O/N (16 h) at 54° C. (20° C. below probes melting temperature) in a humid chamber. Following hybridization, sections were washed 3 times with 0.2×SCC/20 min/60° C. and equilibrated in TN buffer (100 mM Tris-HCl pH 7.5, 150 mM NaCl) for 5 minutes at room temperature. Sections were then blocked in TNB buffer (0.5% Blocking Reagent-Perkin Elmer in TN buffer) for 30 minutes at room temperature. To quench peroxidase activity, the slides were incubated in 3% H2O2 for 1 hr and washed with TNT (0.05% Tween20 in TN buffer) 3×5 min. Diluted anti-DIG-POD 1:500 in TNB was applied and incubated for 30 min at room temperature. After 3 washes with TNT—3×5 min slides were incubated 10 min at room temperature with diluted Cy3-Tyramide 1:50 in Amplification Reagent (TSATM Plus Cy5 Fluorescence System, Perkin Elmer). Finally slides were washed with TNT—3×5 min and mounted in Mowiol.

LNA probes were designed by Exiqon:

miR-21-3p:
/5DigN/GACAGCCCATCGACTGCACTGCTGTTG/3Dig_N/
(SEQ ID NO: 26 with Dig labelling at 5′ and 3′)
Scramble-miR:
/5DigN/GTGTAACACGTCTATACGCCA/3Dig_N/
(SEQ ID NO: 27 with Dig labelling at 5′ and 3′)

Abdominal Skin Ex-Vivo Culturing Conditions:

Immediately after surgery, human abdominal skin was cleaned from its adipose tissue and 0.5 mm² biopsies were deposed on adjusted agars-based-bed filled dishes and incubated in humidified incubator at 37° C. with 5% CO₂ for 24 hours. Topical application of the miR-21-3p-based formulation was performed 24 hours and 2 hours before UV irradiation, immediately and 2 hours after UV irradiation. Skin was processed for dermis-epidermis separation prior to RNA extraction 18 hours after UV irradiation. Agars gel contains DMEM Glutamax-I™ media (Gibco Cat No. 31331-028), 20% of FBS and 1% of antibiotic (penicillin/streptomycin). 0.25 g of agars are diluted in 10 ml of water and heated for 1 min (microwave). 1 ml of agars mix is added for each 10 ml of culture media 6 mL of this solution is quickly added to 6 cm petri dishes. When solidified, a “bed” is dug in the agar in which skin biopsy is field. The epidermis remains at surface.

MiR-21-3p Inhibitor Topical Lotion Formulation:

The lotion contains glycerol as a humectant, DMSO, transfection reagent (RNAimax, Invitrogen, catalogue number 13778030) and 50 μM of miR-21-3p LNA-inhibitor diluted in PBS (Exiqon, I-has-miR-21-3p product number 500150, SEQ ID NO: 29: CCCATCGACTGGTGTT) or its control (Exiqon, MM-has-miR-21-3p, SEQ ID NO: 30: CCCTTCGTCAGGTGTA) at the following respective volume proportions: 9:1:1:8. 16 ul of lotion were used by application to each 0.5 mm² biopsies.

Human Tissues Information:

Cutaneous SCC samples were obtained anonymously from the Department of Dermatology, University Hospital of Lausanne, Switzerland. Normal skin was from healthy adult volunteers or from the edges of skin tumors. SCC was diagnosed by experienced pathologists. Informed consent for research was obtained prior to routine diagnostic services. All samples include the dermis and the epidermis.

Cutaneous psoriasis samples were obtained anonymously from the Department of Dermatology, Universitäts Spital Zürich, Switzerland. Psoriasis samples were obtained from adult volunteers and diagnosed by experienced pathologists. Informed consent for research was obtained prior to routine diagnostic services. All samples include the dermis and the epidermis.

Abdominal skin samples were obtained anonymously from the Department of Interne Medicine, University Hospital of Geneva, Switzerland. Informed consent for research was obtained prior to surgery.

Statistics Summary:

Unless indicated otherwise, all data are presented as the means standard errors of the mean, and statistical differences were evaluated by two-tailed Student's t-tests. For all analyses, we considered p value<0.01 to be statistically significant.

Tape Stripping of Mouse Skin:

Back skin of hairless mice was stripped 10 times with ordinary adhesive tape. For each stripping, a fresh piece of tape was lightly pressed onto the skin and pulled off. 24 h after tape stripping, mice were euthanized and epidermal and dermal sheets were separated and followed with RNA extraction of epidermis samples.

### Example 1. PPARβ/δ Regulates the Expression of UV-Induced Epidermal miR-21-3p, a miRNA Also Up Regulated in Human Skin Carcinomas

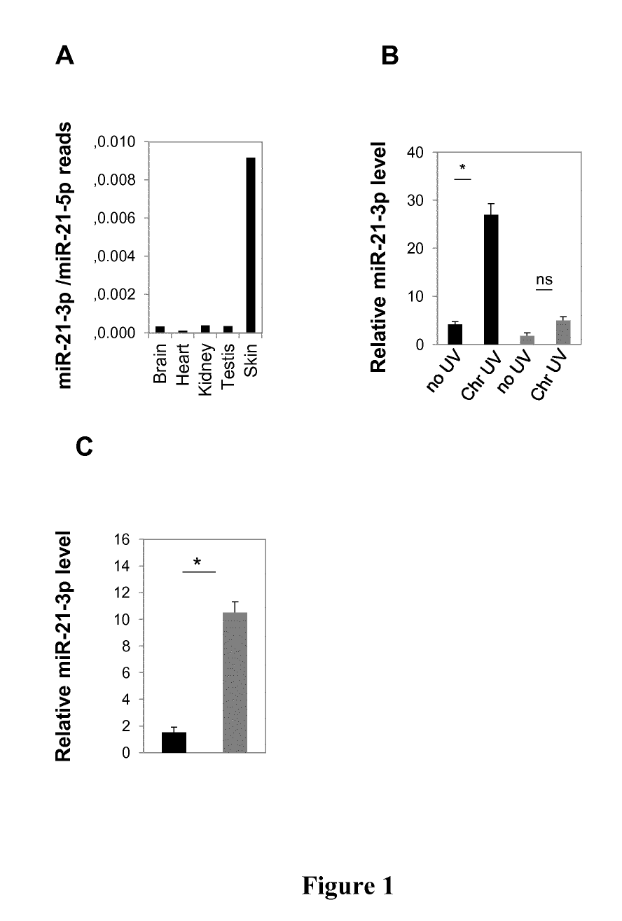

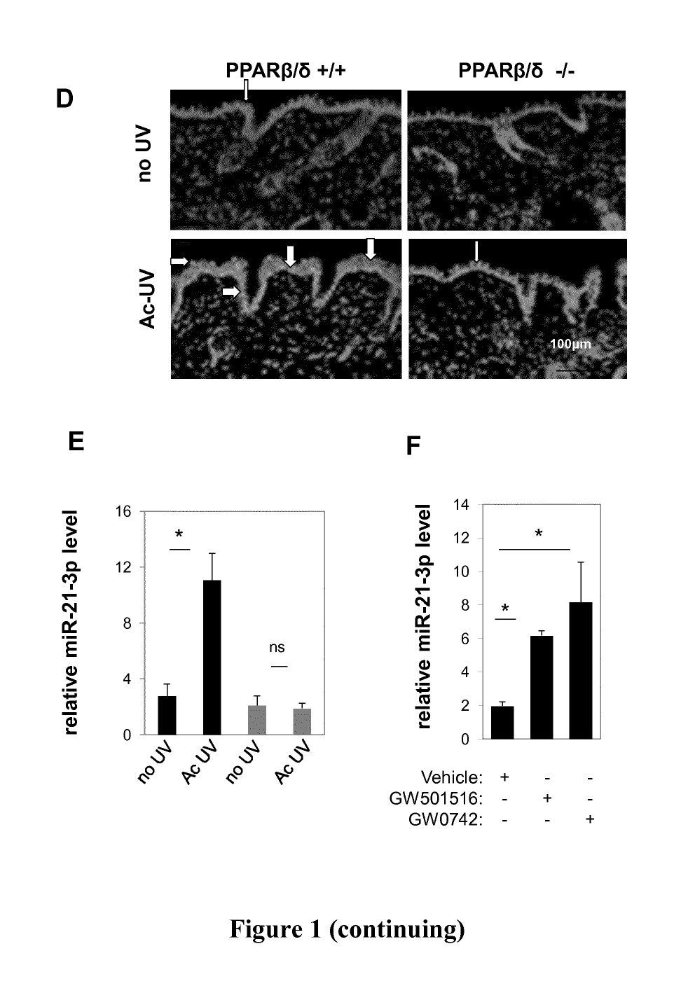

MiR-21-3p is the passenger miRNA of miR-21-5p (commonly named miR-21), a well-characterized “oncomiR”. Given the known induction of miR-21-5p by UV irradiation and its oncogenic role in skin squamous cell carcinomas, we tested the hypothesis that in the skin, miR-21-3p is not degraded (as passenger miRNAs are commonly thought to be) by quantifying the miR-21-3p/miR-21-5p ratio using RNA sequencing counts in various murine organs, including unchallenged skin. This ratio was strikingly higher in the skin compared to kidney, testis, brain and heart (FIG. 1A). Although miR-21-3p expression remained modest compared to miR-21-5p, these data show that miR-21-3p was enriched over miR-21-5p in the skin specifically, pointing to a selective importance for miR-21-3p in the physiology of this organ. Consistent with this hypothesis, we found that miR-21-3p expression was not only up-regulated in response to UV in the skin of PPARβ/δ+/+ mice (FIG. 1B), but was also considerably increased in human squamous cell carcinoma biopsies compared to healthy skin (FIG. 1C).

Further RT-PCR quantification highlighted that the level of miR-21-3p was induced in the chronically UV irradiated skin of PPARβ/δ+/+ mice, while UV exposure failed to up-regulate miR-21-3p level in the skin of PPARβ/δ−/− mice (FIG. 1B). Furthermore, the magnitude of UV-dependent increase of miR-21-3p level was reduced by pharmacological inhibition of PPARβ/δ with its antagonist GSK0660 in PPARβ/δ+/+ skin, showing that PPARβ/δ activity is required for full activation of cutaneous miR-21-3p upon UV exposure (data not shown).

Both the epidermis and the dermis are affected by UV exposure. The epidermis is directly exposed to UVA and B, while the dermis is reached by UVA only and indirectly exposed to UVB-induced changes via the epidermal-dermal cross talk.

In order to distinguish in which skin compartment was the level of miR-21-3p induced by UV exposure, in situ hybridization was performed in PPARβ/δ+/+ and −/− skin samples, and miR-21-3p levels were quantified in isolated epidermis and dermis, which successful separation was monitored using specific markers. The subcellular localization of miR-21-3p in non-irradiated skin revealed a clear preferential expression of this miRNA in the epidermis and hair follicles of PPARβ/δ+/+ skin, with low or no expression in the dermis (FIG. 1D). Following acute UV exposure, the level of miR-21-3p was increased in PPARβ/δ+/+ but not in the PPARβ/δ−/− epidermis, as shown by in situ hybridization (FIG. 1D), and RT-qPCR (FIG. 1E). UV-induced miR-21-3p increase was specific to the epidermis, since its level in the dermis remained unchanged after acute UV irradiation (FIG. 1D). The specific localization of miR-21-3p in the epidermis could be advantageous for therapeutic applications. Of note, the expression pattern of miR-21-3p correlated with the subcellular localization and the changes observed in PPARβ/δ expression levels, which were similarly up-regulated in the epidermis by acute UV exposure, while remaining stable in the dermis. In line with these in vivo data pointing to a specific expression of miR-21-3p in keratinocytes, miR-21-3p level was increased following activation of PPARβ/δ with its agonists GW501516 and GW0742 in the human keratinocyte cell line HaCat (FIG. 1F), like the two well-characterized PPARβ/δ target genes Angpt14 and Tgfβ-1 (data not shown). Collectively, these findings revealed that miR-21-3p expression is under the control of PPARβ/δ, in murine epidermis exposed to chronic or acute UV, as well as in HaCat human keratinocytes. Furthermore, miR-21-3p likely belongs to a skin cancer molecular signature, since its expression is dramatically increased in murine and human squamous cell carcinomas.

### Example 2. MiR-21-3p Mimic Treated Keratinocytes Show a Psoriasis-Like Molecular Signature

Genome-wide microarray analysis of miR-21-3p mimic treated human HaCaT keratinocytes compared to a three-bases mismatching miR-21-3p control sequence indicates that among the mRNA that exhibit a mimic-dependent regulation, 6.7% of them are known psoriasis-related mRNAs (Miridian™, Thermo Scientific Dharmacon, cat number C-30123-01-0005) or control (amino acid sequence of cel-miR-67-3p of SEQ ID NO: 28 (UCACAACCUCCUAGAAAGAGUAGA, Miridian™, Thermo Scientific Dharmacon, cat number CN-001000-01-05) see in material and methods (cell culture, transfection and treatments).

The majority of them are up-regulated upon miR-21-3p mimic transfection and are also known to be up-regulated in psoriasis (Table 1).

These up-regulated mRNA include the mRNA of the cytokine-activated S100A8 protein known to be up-regulated in hyperproliferative and psoriatic epidermis (Nukui et al., 2008, J Cell Biochem, 104(2):453-64); Bracke et al., 2013, Arch Dermatol Res, 305(6):501-512); as well as STAT1 mRNA shown to be up-regulated in psoriasis lesion (Hald et al., 2013, Br J Dermatol. 168(2): 302-10; Bracke et al., 2013 (supra)); or the chemokine mRNA CCL5 known to induce skin-infiltrating T-cells leading to inflammation in psoriasis and other inflammatory skin diseases (Canavese et al., 2010, Dermatol Sci. 58(3):171-6; Bracke et al., 2013 (supra)) or other mRNAs of inflammatory mediators like IL1RN, CXCL10 and CXCL 11 known to be up-regulated in psoriasis and in atopic dermatitis (Giustizieri et al., 2001, J Allergy Clin Immunol. 107(5):871-7; Shepherd, 2004, J Invest Dermatol, 122(3): 665-9; Flier et al., 2001, J Pathol. 194(4): 398-405), suggesting a pro-psoriasis effect for miR-21-3p gain of function in HaCat keratinocytes at the molecular level.

Among the down-regulated mRNA in miR-21-3p mimic treated keratinocytes, Casp14 mRNA, which expression is known to be inhibited in the parakeratotic regions of psoriatic skin (Lippens et al., 2000, Cell Death Differ. 7(12):1218-24; Bracke et al., 2013 (supra)), suggesting a pro-psoriasis role for miR-21-3p gain of function in HaCat keratinocytes.

TABLE 1

| Fold | | | | | |
|---|---|---|---|---|---|
| change | | | | | |
| mimic/ | | Gene | | Psoriasis | |
| control | P value | name | Gene description | involvement | Reference |
| −1.598 | 2.97E−04 | CASP14 | Caspase 14, | down- | Bracke et al, 2013, |
| | | | apoptosis-related | regulated | supra); |
| | | | cysteine peptidase | in psoriasis | Lippens et al, |
| | | | | | 2000 (supra) |
| 1.733 | 4.55E−02 | UGT2B7 | UDP glucuronosyl | cornification | Jabbari et al., |
| | | | transferase 2 family, | markers | 2012, J Invest |
| | | | polypeptide B7 | | Dermatol., |
| | | | | | 132(1): 246-9 |
| 1.752 | 4.21E−03 | KRT1 | Keratin 1 | up-regulated | Roth et al, 2012, |
| | | | | in psoriasis | J Cell Sci, |
| | | | | | 125: 5269-79 |
| 1.863 | 1.23E−05 | EPGN | Epithelial mitogen | up-regulated | Jabbari et al, |
| | | | homolog (mouse) | in psoriasis | 2012, (supra) |
| 1.968 | 8.71E−04 | S100A8 | S100 calcium | up-regulated | Bracke et al, 2013 |
| | | | binding protein A8 | in psoriasis | (supra); Nukui et |
| | | | | | al, 2008 (supra |
| 1.984 | 5.04E−07 | STAT1 | Signal transducer | up-regulated | Bracke et al, 2013 |
| | | | and activator of | in psoriasis | (supra) |
| | | | transcription | | |
| 2.093 | 4.29E−07 | CCL5 | Chemokine 5 | up-regulated | Bracke et al, |
| | | | | in psoriasis | 2013, supra); |
| | | | | | Canavese et al, |
| | | | | | 2010 (supra) |
| 2.138 | 2.60E−07 | CD274 | CD274 molecule | up-regulated | Jabbari et al, |
| | | | | in psoriasis | 2012, (supra) |
| 2.320 | 3.74E−08 | IL1RN | Interleukin 1 | up-regulated | Giustizieri et al, |
| | | | receptor antagonist | in psoriasis | 2001 (supra); |
| | | | | | Shepherd et al, |
| | | | | | 2004 (supra) |
| 2.465 | 5.46E−09 | TNFSF10 | Tumor necrosis | up-regulated | Peternel et al, |
| | | | factor (ligand) | in psoriasis | 2011, Arch |
| | | | superfamily, | | Dermatol Res, |
| | | | member 10 | | 303(6): 389-97 |
| 4.257 | 4.66E−06 | CXCL11 | Chemokine ligand | up-regulated | Flier et al, 2001, |
| | | | 11 | in psoriasis | J Pathol. 194(4): |
| | | | | | 398-405 |
| 7.165 | 3.44E−06 | CXCL10 | Chemokine ligand | upregulated | Giustizieri et al, |
| | | | 10 | in psoriasis | 2001 (supra); |
| | | | | | Bracke et al, 2013 |
| | | | | | (supra) |

### Example 3. MiR-21-3p Mimic Treated Keratinocytes Show a Similar Expression Profile of Some Markers Mentioned in Table 1 as in Various Skin Disorders

Regarding the involvement of some of the markers indicated in Table 1, the expression of which is modulated by miR-21-3p, in various skin disorders, it can be mentioned that:
Casp14 deficient newborn mice shows barrier disruption, delay in cornification and high UV-sensitivity and are more prone to the development of parakeratosis (Hoste et al., 2013, J Invest Dermatol, 133(3):742-50). This also suggests a role for CASP14 in the maintenance of the skin barrier integrity that is strongly in cause in eczema, acne and other inflammatory skin diseases including as well psoriasis and in UV photodamage protection.
KRT1, a major constituent of the intermediate filament cytoskeleton in suprabasal epidermis, is mutated in epidermolytic ichthyosis in humans. Indeed, as described in Roth et al. (2012, J Cell Sci, 125(Pt 22):5269-79), the “absence of Krt1 causes a prenatal increase in interleukin-18 and the S100A8 and S100A9 proteins, accompanied by a barrier defect and perinatal lethality”. It is also shown that transcriptome profiling revealed a Krt1-mediated gene expression signature similar to atopic eczema and psoriasis, suggesting a functional link between KRT1 and human inflammatory skin diseases.
The S100A8 protein has also been shown to be involved in skin tumor formation (McNeill et al., 2014, Int J Cancer, January 16; Hummerich et al, 2006, Oncogene, 25(1):111-21).
COX2 (PTGS2) has also been linked to a large range of inflammatory skin diseases including Vitiligo (Li et al., 2009, J Dermatol Sci, 53(3):176-81), leprosy (Pesce et al., 2006, Am J Trop Med Hyg, 74(6):1076-7).
MMP1 has been associated with wound healing of the skin (Stevens et al., 2012, Mol Biol Cell, 23(6):1068-79).

Together, these data point to a stimulatory effect of miR-21-3p on a large panel of inflammation-related markers involved in multiple skin disorders including skin cancers, psoriasis, leprosy, vitiligo, eczema, keratosis, epidermolytic ichthyosis, UV photodamages, as well as in skin inflammatory reactions like acne, allergy, UV erythema, skin ageing.

### Example 4. Inflammatory Markers are Up-Regulated in miR-21-3p Mimic Treated HaCat Keratinocytes

COX2 (prostaglandin-endoperoxide synthase also PTGS) is an enzyme responsible for the formation of biological mediators called prostanoids (prostaglandins, prostacyclin and thromboxane). The mRNA of COX2 has been shown to be up-regulated in psoriatic skin (Yalcin et al., 2007, Anal Quant Cytol Histol, 29(6): 358-64).

IL-6 production has been associated with psoriatic skin (Grossman et al., 1989, Proc Natl Sci USA, 86(16):6367-71).

Real-time qPCR quantification of Cox2 and IL-6 mRNA shows up-regulated expression of these two inflammatory markers in miR-21-3p mimic treated HaCat cells compared to control treated cells (FIG. 2A, B) (Miridian™, Thermo Scientific Dharmacon, cat number C-30123-01-0005) or control (amino acid sequence of cel-miR-67-3p of SEQ ID NO: 28 (UCACAACCUCCUAGAAAGAGUAGA, Miridian™, Thermo Scientific Dharmacon, cat number CN-001000-01-05) see in material and methods (cell culture, transfection and treatments).

Thus, these observations indicate a possible pro-inflammatory role for miR-21-3p in keratinocytes.

### Example 5. Phosphatidylcholine Content is Up-Regulated in miR-21-3p Mimic Treated HaCat Keratinocytes

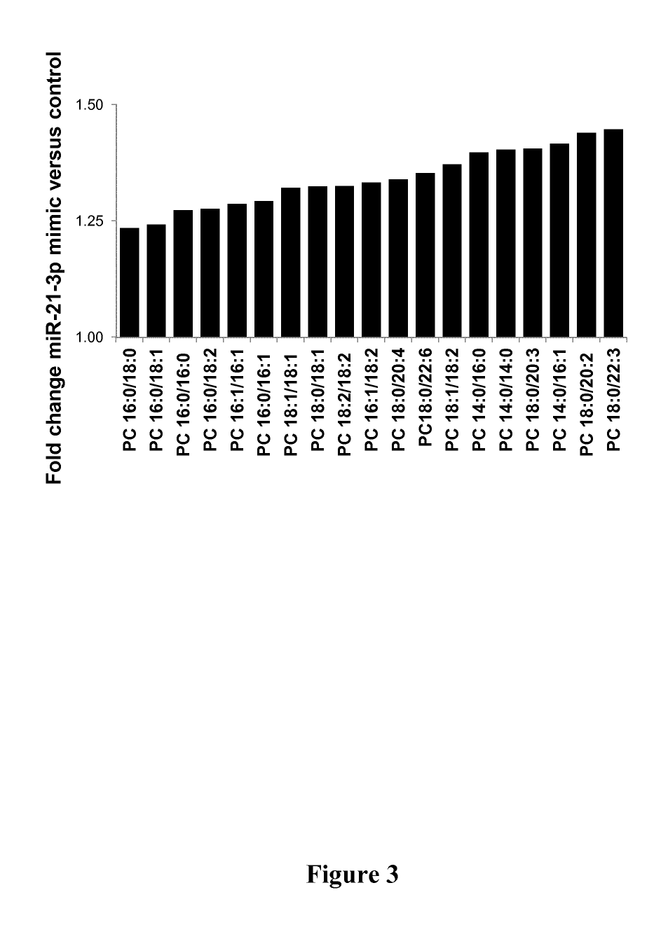

Quantification of phosphatidylcholine content in miR-21-3p mimic treated HaCat cells compared to control treated HaCat cells (Miridian™, Thermo Scientific Dharmacon, cat number C-30123-01-0005) or control (amino acid sequence of cel-miR-67-3p of SEQ ID NO: 28 (UCACAACCUCCUAGAAAGAGUAGA, Miridian™, Thermo Scientific Dharmacon, cat number CN-001000-01-05) see in material and methods (cell culture, transfection and treatments) indicates an overall increased level ofphosphatidylcholine upon miR-21-3p gain of function (FIG. 3).

Previous studies showed an increase in phosphatidylcholine content in psoriatic papules compared to normal skin (Sergeev et al., 1993, Biull Eksp Biol Med., 116(9): 271-2). This observation suggests that miR-21-3p gain of function induces an increase in phosphatidylcholine in HaCat cells similar to what is observed in psoriatic skin.

### Example 6. The Cell Cycle Inhibitors p15 and p21 are Down Regulated in miR-21-3p Mimic Treated HaCat Keratinocytes

Cdkn2B (p15) and CdknlA (p21) are two cyclin-dependent kinase inhibitors responsible for the TGFβ-1-induced cell cycle arrest.

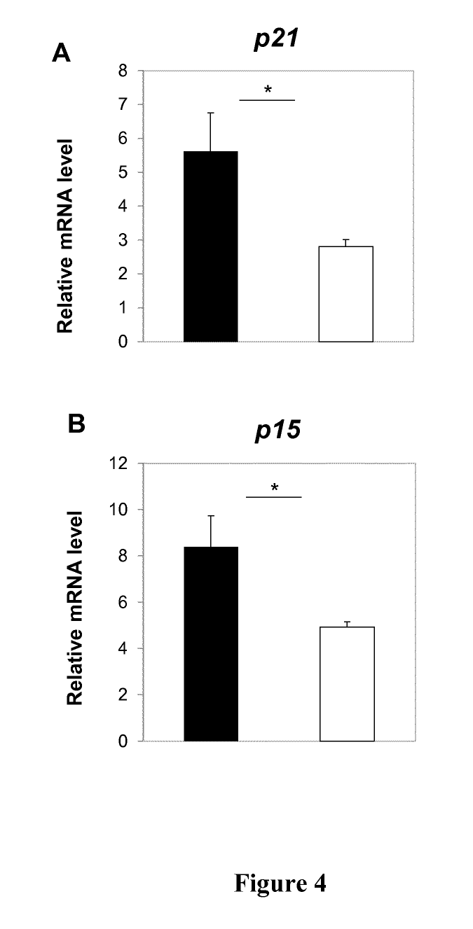

Real-time qPCR quantification of the mRNA of these cell cycle inhibitors p15 and p21 shows that they are down-regulated in miR-21-3p mimic treated HaCat keratinocytes compared to control (FIG. 4A, B).

These observations suggest that miR-21-3p gain of function may stimulate cell cycle. Inhibition of cell cycle inhibitors may lead to skin hyperproliferation observed in psoriasis phenotype.

### Example 7. MMP1 is Down-Regulated in miR-21-3p Mimic Treated HaCat Keratinocytes Compared to Control

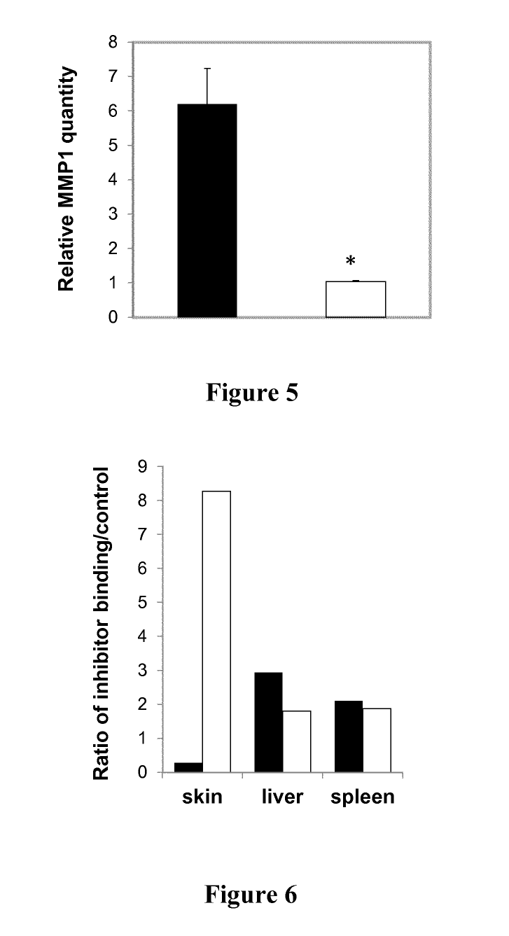

Real-time qPCR quantification of Mmp1 mRNA shows that it is down-regulated in miR-21-3p mimic treated HaCat cells compared to control (FIG. 5).

Mmp1 expression has been show as down-regulated in lesional scales of patients with severe psoriasis (Flisiak et al., 2006, Acta Derm Venereol, 86(1): 17-21).

Altogether, it points to the possibility of down-regulating levels of Mmp1 by influencing levels/activity of miR-21-3p.

### Example 8. MiR-21-3p Inhibitor Fluorescence Increases in the Skin Upon UV-Induced Inflammatory Condition

Efficient delivery of a FAM-labelled LNA-mmu-miR-21-3p inhibitor (Exiqon Inhibitor Probe Number 199900, SEQ ID NO: 7: CCATCGACTGCTGTT) or in vivo inhibitor 3 mismatch control “mmu-miR-21a-3p_3MM, 5′-FAM labelled” as a control (SEQ ID NO: 9: CCCTAGACTGCTCTT), aimed at counteracting the endogenous miR-21-3p, was monitored using the FAM fluorescence, supposed to be stabilized upon binding of the inhibitor to its target. Quantification of the fluorescence in skin, liver, spleen and muscle lysate showed that the fluorescence was decreased in liver, spleen and muscle, whereas was specifically increased in the skin in acutely irradiated mice in comparison with control treated mice, suggesting that miR-21-3p inhibitor was stabilized in this organ after acute UV irradiation (FIG. 6).

As shown in FIG. 6, miR-21-3p inhibitor fluorescence increases in the skin upon UV-induced inflammatory condition. This also addresses the skin-specificity of the miR-21-3p inhibitor delivery, which accumulates in the inflamed skin rather than in spleen or liver, and would thus make miR-21-3p inhibitors advantageous as therapeutics to avoid or reduce a systemic effect and unwanted side effects.

### Example 9. Gain of miR-21-3p Function Causes Changes in Inflammation Pathways in HaCaT Human Keratinocytes

In order to characterize the role of miR-21-3p in keratinocytes, HaCaT cells were transfected with a miR-21-3p mimic oligonucleotide sequence and a scramble sequence as a control (Miridian™, Thermo Scientific Dharmacon, cat number C-30123-01-0005) or control (amino acid sequence of cel-miR-67-3p of SEQ ID NO: 28 (UCACAACCUCCUAGAAAGAGUAGA, Miridian™, Thermo Scientific Dharmacon, cat number CN-00 1000-01-05) see in material and methods (cell culture, transfection and treatments).

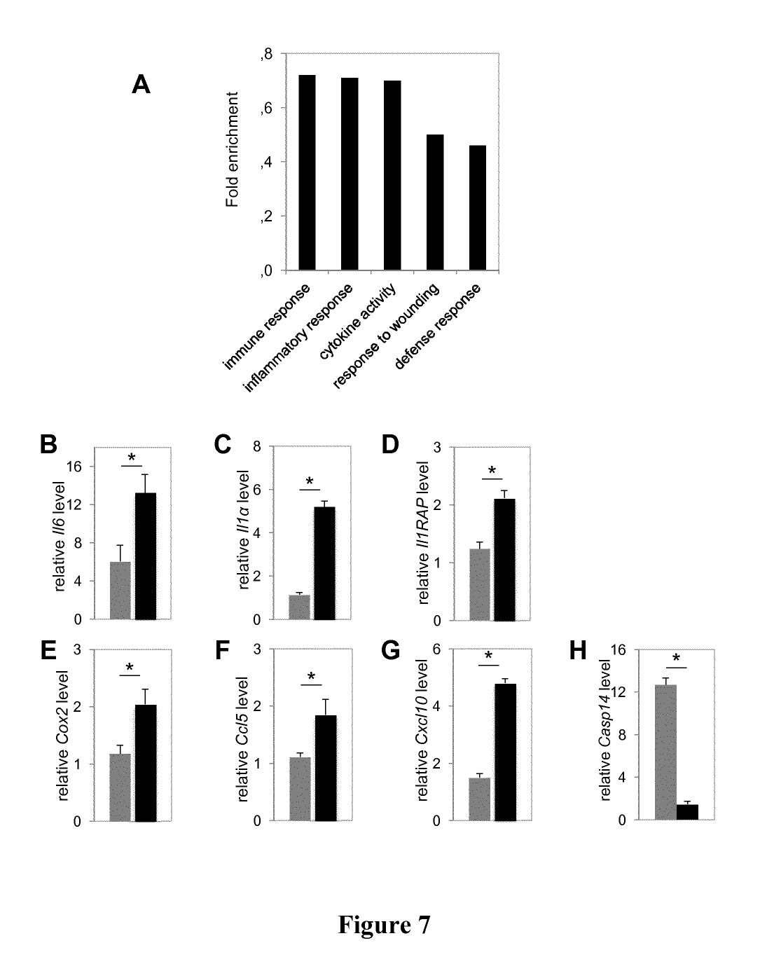

The global impact of miR-21-3p gain-of-function on mRNA expression was addressed using a microarray analysis, which highlighted the immune and inflammation response pathways as of the most affected biological functions (FIG. 7A). Consistent with this analysis, miR-21-3p gain-of-function in HaCaT cells provoked a strong up-regulation of the pro-inflammatory cytokines IL6 and IL1α, of the IL receptor co-factor IL1RAP, of the prostanoid-producing enzyme Cox2, and of the chemokines Ccl5 and Cxcl10 (FIG. 7B-G). Interestingly, epidermis specific Caspase 14 (Casp14) expression, which down-regulation has been previously associated with UV irradiation and inflammatory skin diseases (Hoste et al., 2013, Journal of Investigative Dermatology 133, 742-750; Denecker et al., 2007, Nature Cell Biology, 9(6): 666-674) was significantly reduced in miR-21-3p mimic compared to control treated cells (FIG. 7H).

Therefore, this data further support the role of miR-21-3p in mediating immune and inflammatory response in the skin.

### Example 10. Measurement of miR-21-3p in a Non-Inflammatory Transient Epidermal Barrier Disruption Tape Stripping Murine Skin Model

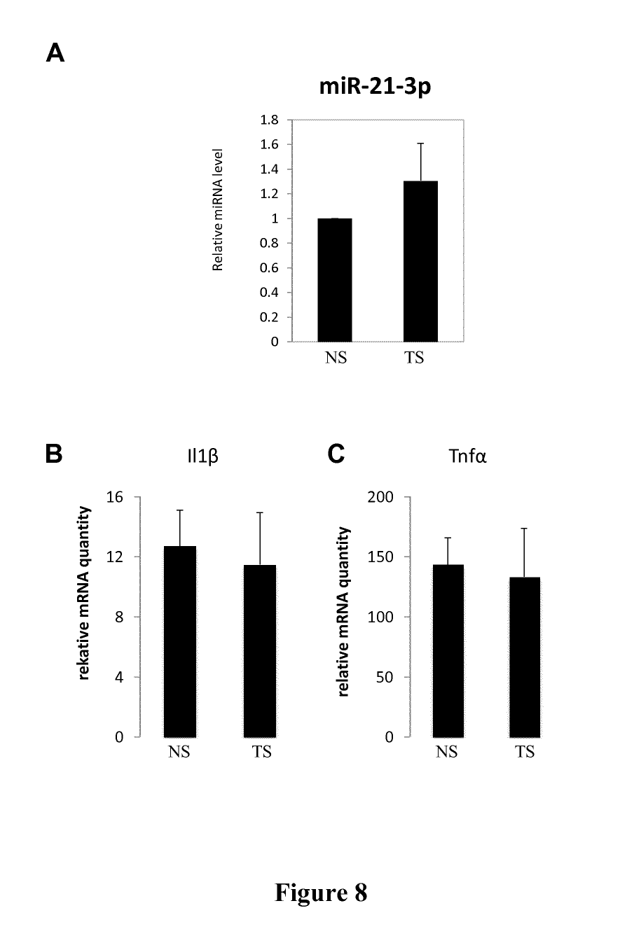

In order to evaluate the specificity of miR-21-3p activation in regard to inflammatory situation, a non-inflammatory transient epidermal barrier disruption was performed by tape stripping on murine skin. In such conditions, miR-21-3p was not up-regulated in comparison to control skin (FIG. 8A) as observed for inflammatory markers (FIG. 8B, C).

These results suggest that miR-21-3p up-regulation does not occur in moderate and non-cutaneous inflammatory conditions. Therefore, inflammatory stress is required for miR-21-3p up-regulation to occur.

### Example 11. Preventive Anti-Inflammatory Effect of Human miR-21-3p Inhibitor after UV Exposure of Ex Vivo Cultured Human Abdominal Skin

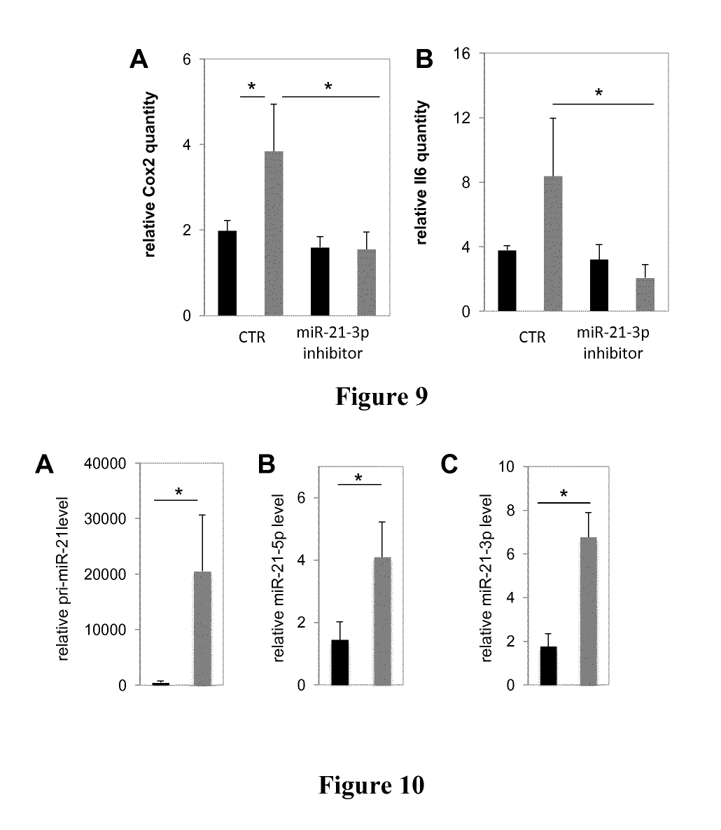

The effect of a miR-21-3p preventive inhibition was evaluated in case of cutaneous inflammation. Human skin topically treated, ex vivo, with an LNA-anti-miR-21-3p formulation after having been exposed to acute UV (grey; no UV in black), showed a reduction of IL6 and Cox2 expression in comparison with LNA-control treated biopsies (FIG. 9A, B). Formulations are described in material and methods (MiR-21-3p inhibitor topical lotion formulation). Inhibitor and controls are: Exiqon, I-has-miR-21-3p product number 500150, SEQ ID NO: 29: CCCATCGACTGGTGTT) or its control (Exiqon, MM-has-miR-21-3p, SEQ ID NO: 30: CCCTTCGTCAGGTGTA) These data demonstrate the strong clinical potential of miR-21-3p inhibitor-based therapies dedicated to preventive treatment of cutaneous inflammation.

### Example 12. MiR-21-3p is Up-Regulated Under Skin Inflammatory Conditions: Human Psoriasis Biopsies

The observation that miR-21-3p gain of function was associated with a pro-inflammatory phenotype in human keratinocytes in culture (Example 9) raises the exciting possibility that miR-21-3p inhibitors may be used as therapeutic anti-inflammatory agents.

In agreement with a role of miR-21-3p in the up-regulation of skin inflammation, quantification of miR-21-3p expression level was significantly increased in cutaneous inflammatory conditions including patient psoriasis biopsies compared to normal skin (FIG. 10 A-C).

### Example 13. MiR-21-3p Levels During Melanoma Development

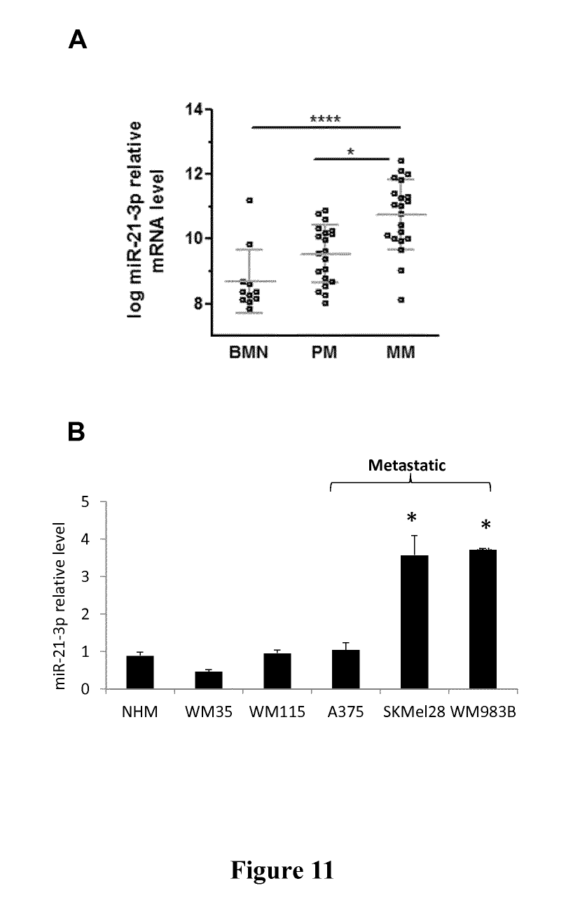

It was observed that miR-21-3p level was increased in in primary melanoma (PM) and in malignant melanoma (MM) compared to benign melanocytic naevus (BMN), (FIG. 11A) and was increased in two melanoma metastatic cell lines (SKMel28 and WM983B) compared with normal and with non-metastatic melanoma cell lines (WM35, WM115 and A375) (FIG. 11B).

These data suggest the involvement of miR-21-3p in melanoma development and/or malignant cell migration and/or invasiveness. Thus, modulation of miR-21-3p by administration of miR-21-3p inhibitors could allow treatment of skin cancers.

### Example 14. Identification of Direct Target of miR-21-3p

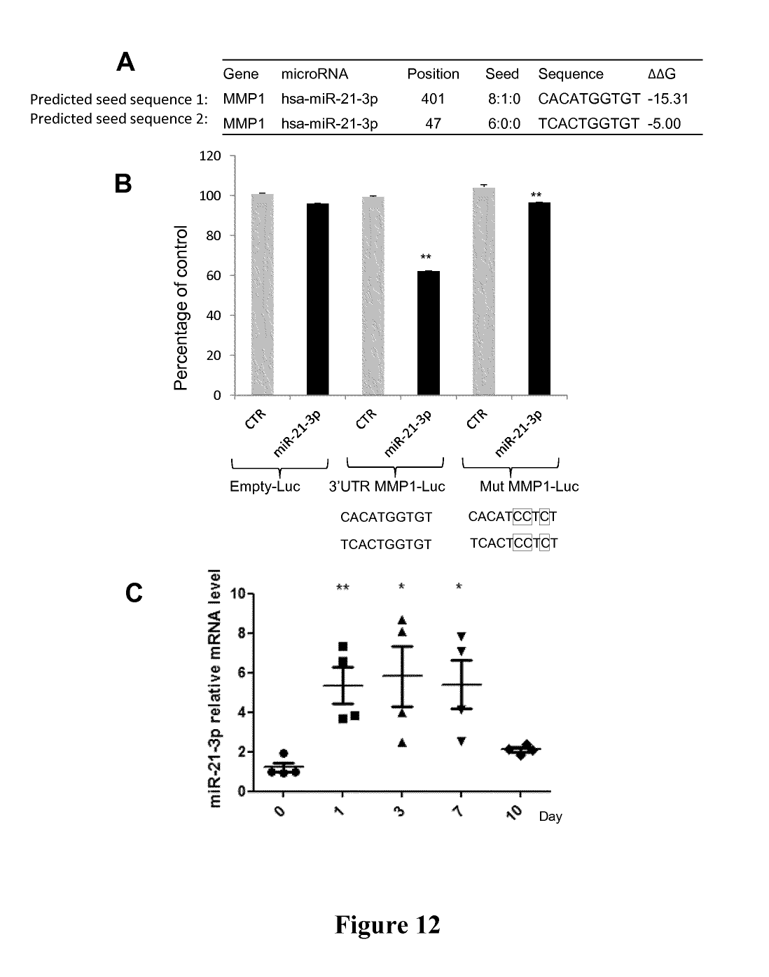

Combined analysis of microarray and in silico miRNA target prediction indicated that MMP1 mRNA could be a direct miR-21-3p target gene with two predicted miR-21-3p binding sites on MMP1 3′UTR (FIG. 12A). This hypothesis was further confirmed in human keratinocytes where the co-transfection with the 3′UTR-MMP1-luciferase-plasmid and a miR-21-3p mimic (FIG. 12B) resulted in the down-regulation of the 3′UTR-MMP1-luciferase signal in comparison with scramble control treated cells and with the 3′UTR-MMP1 mutated-luciferase signal. Matrix metalloproteinases (MMPs) up-regulation facilitates aging and cancer, miR-21-3p-mimic-based products that inhibit MMP1 (Philips et al., 2011, Enzyme Research) could be of interest in prevention or treatment of aging and skin cancer. Indeed, miR-21-3p was up-regulated in malignant melanoma and in metastatic melanoma cells (FIG. 11A). Moreover, inflammation is involved in skin ageing process as well. MiR-21-3p expression level was measured before (day 0), during (day 1 to day 7), and after (day 10), the skin wound healing process. Interestingly miR-21-3p expression levels increased along this process and decreased when the wound has healed (FIG. 12C).

Together, with miR-21-3p pro-inflammatory properties (FIGS. 2 and 7) and its inhibitory action on MMP1, miR-21-3p inhibition could have a medical and cosmetic interest in the context of skin cancer, skin injury, skin ageing and in addition of the previously described inflammatory skin diseases.

Sequence listing

Human miR-21-3p (miRBase accession number: MIMAT0004494)
SEQ ID NO: 1: CAACACCAGUCGAUGGGCUGU
Murine miR-21-3p (miRBase Accession number MIMAT0004628).
SEQ ID NO: 2: CAACAGCAGUCGAUGGGCUGUC
Inhibitor, complementary sequence of human miR-21-3p (21 nucleotides in total)
SEQ ID NO: 3: ACAGCCCATCGACTGGTGTTG
Human miR-21-3p inhibitor 1 (15 nucleotides in total)
SEQ ID NO: 4: CCATCGACTGGTGTT
Human miR-21-3p inhibitor 2 (18 nucleotides in total)
SEQ ID NO: 5: AGCCCATCGACTGGTGTT
Inhibitor, complementary sequence of murine miR-21-3p (21 nucleotides in total)
SEQ ID NO: 6: ACAGCCCATCGACTGCTGTTG
Murine miR-21-3p inhibitor 1 (15 nucleotides in total)
SEQ ID NO: 7: CCATCGACTGCTGTT
Murine miR-21-3p inhibitor 2 (18 nucleotides in total)
SEQ ID NO: 8: AGCCCATCGACTGCTGTT
in vivo inhibitor mismatch control ″mmu-miR-21a-3p 3MM, 5′-FAM labeled″
SEQ ID NO: 9: CCCTAGACTGCTCTT, with FAM labelling in 5′
Primers for mRNA reverse transcription and quantitative PCR:
mmu-Gapdh mRNA forward primer: SEQ ID NO: 10:
GTATGACTCCACTACGGCAAA
mmu-Gapdh mRNA reverse primer SEQ ID NO: 11: TTCCCATTCTCGGCTTG
mmu-Ef1a1 mRNA forward primer: SEQ ID NO: 12: CCTGGCAAGCCCATGTGT
mmu-Ef1a1 mRNA reverse primer: SEQ ID NO: 13:
TCATGTCACGAACAGCAAAGC
mmu-Pparβ/δ mRNA forward primer: SEQ ID NO: 14: CGGCAGCCTCAACATGG
mmu-Pparβ/δ mRNA reverse primer: SEQ ID NO: 15:
AGATCCGATCGCACTTCTCATAC
hsa-Pparβ/δ mRNA forward primer: SEQ ID NO: 16:
GCATGAAGCTGGAGTACGAGAAG
hsa-Pparβ/δ mRNA reverse primer: SEQ ID NO: 17: GCATCCGACCAAAACGGATA
mmu-Pparβ/δ mRNA forward primer: SEQ ID NO: 18: CGGCAGCCTCAACATGG
mmu-Pparβ/δ mRNA reverse primer: SEQ ID NO: 19:
AGATCCGATCGCACTTCTCATAC
hsa-P21 mRNA forward primer: SEQ ID NO: 20: CTGTCACTGTCTTGTACCCT
hsa-P21 mRNA reverse primer: SEQ ID NO: 21: GGTAGAAATCTGTCATGCTGG
hsa-P15 mRNA forward primer: SEQ ID NO: 22: CGTGGGAAAGAAGGGAAGAGT
hsa-P15 mRNA reverse primer: SEQ ID NO: 23: CCCCAGACGCGCAGC
hsa-pri-miR-21 forward primer: SEQ ID NO: 24: TTTTGTTTTGCTTGGGAGGA
hsa-pri-miR-21 reverse primer: SEQ ID NO: 25: AGCAGACAGTCAGGCAGGAT
miR-21-3p LNA probe: SEQ ID NO: 26: GACAGCCCATCGACTGCACTGCTGTTG,
with Dig labelling in 5′ and 3′
Scramble-miR LNA probe: SEQ ID NO: 27: GTGTAACACGTCTATACGCCA, with
Dig labelling in 5′ and 3′
mi-R-67-3p:
SEQ ID NO: 28: UCACAACCUCCUAGAAAGAGUAGA
Human miR-21-3p inhibitor ″I-has-miR-21-3p″ (16 nucleotides in total)
SEQ ID NO: 29: CCCATCGACTGGTGTT
Human miR-21-3p mismatch control ″MM-has-miR-21-3p″ (16 nucleotides in total)
SEQ ID NO: 30: CCCTTCGTCAGGTGTA

1. A method of preventing and/or treating inflammatory skin disorders, said method comprising administering in a subject in need thereof a miR-21-3p inhibitor or a pharmaceutical formulation thereof.

2. A method according to claim 1, wherein the miR-21-3p inhibitor is an oligonucleotide hybridizing to:
(i) the nucleic acid sequence of miR-21-3p or a fragment thereof, in particular human miR-21-3p of SEQ ID NO: 1 or murine miR-21-3p of SEQ ID NO: 2 or a variant thereof or a fragment thereof, and/or
(ii) the seed region of miR-21-3p, in particular a sequence corresponding to nucleotides 2 to 8 of SEQ ID NO: 1 or nucleotides 2 to 8 of SEQ ID NO: 2 or a variant thereof, and/or
(iii) a fragment of at least 10 contiguous nucleotides from the nucleic acid sequence of miR-21-3p, in particular human miR-21-3p of SEQ ID NO: 1 or murine miR-21-3p of SEQ ID NO: 2 or a variant thereof, optionally comprising the seed region of miR-21-3p, in particular a sequence corresponding to nucleotides 2 to 8 of SEQ ID NO: 1 or nucleotides 2 to 8 of SEQ ID NO: 2 or a variant thereof.

3. A method according to claim 1, wherein the miR-21-3p inhibitor is an oligonucleotide complementary to:
(i) the nucleic acid sequence of miR-21-3p or a fragment thereof, in particular human miR-21-3p of SEQ ID NO: 1 or murine miR-21-3p of SEQ ID NO: 2 or a variant thereof or a fragment thereof, and/or
(ii) the seed region of miR-21-3p, in particular a sequence corresponding to nucleotides 2 to 7 of SEQ ID NO: 1 or nucleotides 2 to 7 of SEQ ID NO: 2 or a variant thereof, and/or
(iii) a fragment of at least 10 contiguous nucleotides from the nucleic acid sequence of miR-21-3p, in particular human miR-21-3p of SEQ ID NO: 1 or murine miR-21-3p of SEQ ID NO: 2 or a variant thereof, optionally comprising the seed region of miR-21-3p, in particular a sequence corresponding to nucleotides 2 to 7 of SEQ ID NO: 1 or nucleotides 2 to 7 of SEQ ID NO: 2 or a variant thereof.

4. A method according to claim 1, wherein the miR-21-3p inhibitor is an oligonucleotide comprising any one of:
(i) SEQ ID NO: 3, or a variant thereof, or a fragment thereof optionally comprising nucleotides 15 to 20 of SEQ ID NO: 3,
(ii) a fragment of at least 10 contiguous nucleotides from SEQ ID NO: 3 optionally comprising nucleotides 15 to 20 of SEQ ID NO: 3,
(iii) SEQ ID NO: 4 or SEQ ID NO: 5,
(iv) SEQ ID NO: 6, or a variant thereof, or a fragment thereof optionally comprising nucleotides 15 to 20 of SEQ ID NO: 6,
(v) a fragment of at least 10 contiguous nucleotides from SEQ ID NO: 6 optionally comprising nucleotides 15 to 20 of SEQ ID NO: 6,
(vi) SEQ ID NO: 7 or SEQ ID NO: 8.

5. A method according to claim 1, wherein the miR-21-3p inhibitor is an oligonucleotide comprising a nucleotide sequence of SEQ ID NO: 29.

6. A method according to claim 1, wherein the miR-21-3p inhibitor is a modified oligonucleotide such as a LNA-modified oligonucleotide and/or wherein said oligonucleotide is chemically linked to cholesterol.

7. A method according to claim 1, wherein said inflammatory skin disorder is selected from psoriasis, dermatitis such as ectopic dermatitis, acne, rosacea, Stevens-Johnson syndrome, toxic epidermal necrolysis, systemic lupus erythematous, skin allergies, atopic eczema, parakeratosis, keratosis, skin cancers such as squamous cell carcinoma, basal cell carcinomas and melanomas, leprosy, vitiligo, epidermolytic ichthyosis, UV photodamages, topical allergy, UV erythema, skin ageing following UV exposure, wounds, local inflammation following injury or wounds such as bump or hematoma, non-pre-malignant or pre-malignant structure affecting the skin such as actinic keratosis and seborrheic keratosis.

8. A method according to claim 1, wherein said inflammatory skin disorder is psoriasis or dermatitis.

9. A method according to claim 1, wherein said skin disorder is keratosis or a skin cancer.

10. (canceled)

11. A method according to claim 1, wherein the miR-21-3p inhibitor comprises a nucleotide of SEQ ID NO: 3 or a fragment of SEQ ID NO: 3, optionally comprising nucleotides 15 to 20 of SEQ ID NO: 3.

12. An ex-vivo method of prognosis and/or diagnosis of a skin disorder in a subject comprising:
a) Providing a biological sample from a subject;
b) Determining the level of miR-21-3p, in said sample;
c) Comparing the level of miR-21-3p determined in step b) with the level of miR-21-3p in a control sample:
wherein an increased level of miR-21-3p determined in step b) compared to the control sample is indicative of the subject being at risk for developing, or having, a skin disorder.

13. A miR-21-3p inhibitor having an oligonucleotide sequence consisting of any one of:
(i) a fragment of SEQ ID NO: 3 or a variant thereof, optionally comprising nucleotides 15 to 20 of SEQ ID NO: 3,
(ii) SEQ ID NO: 4 or SEQ ID NO: 5 or a variant thereof, or a fragment thereof,
(iii) SEQ ID NO: 6, or a variant thereof, or a fragment thereof optionally comprising nucleotides 15 to 20 of SEQ ID NO: 6,
(iv) a fragment of SEQ ID NO: 6 optionally comprising nucleotides 15 to 20 of SEQ ID NO: 6,
(v) SEQ ID NO: 7 or SEQ ID NO: 8,
(vi) SEQ ID NO: 29, or a variant thereof, or a fragment thereof,

14. A miR-21-3p inhibitor according to claim 13, wherein said oligonucleotide is a LNA-modified oligonucleotide, wherein the ribose ring of one or more nucleotides of said LNA-modified oligonucleotide is locked by a methylene bridge connecting the 2′-O atom and the 4′-C atom and/or wherein said oligonucleotide is chemically linked to cholesterol.

15. (canceled)

16. A composition comprising a miR-21-3p inhibitor according to claim 13 or a vector comprising a nucleic acid encoding said miR-21-3p inhibitor, and a pharmaceutically acceptable carrier or a cosmetically acceptable carrier.

17. A composition according to claim 16 wherein said composition is a cosmetic composition.

18. A composition according to claim 16, wherein said miR-21-3p inhibitor is a fragment of SEQ ID NO: 3 of 2 to 16 nucleotides in length such as 2 to 8 nucleotides in length optionally comprising nucleotides 15 to 20 of SEQ ID NO: 3.

19. A composition according to claim 16, wherein the composition is a topical formulation.

20. (canceled)

21. (canceled)

22. (canceled)

23. (canceled)

24. A composition according to claim 17, wherein said miR-21-3p inhibitor is a fragment of SEQ ID NO: 3 of 2 to 16 nucleotides in length such as 2 to 8 nucleotides in length optionally comprising nucleotides 15 to 20 of SEQ ID NO: 3.

25. A composition according to claim 17, wherein the composition is a topical formulation.

26. A method according to claim 1, wherein said miR-21-3p inhibitor is administered topically to said patient in the form of composition of claim 16.
